(19) United States
(12) Patent Application Publication
(10) Pub. No.: US 2004/0192641 A1
(43) Pub. Date: Sep. 30, 2004

(54) Special preparation of anticancer drugs made by novel nanotechnology

(76) Inventor: Yaguang Liu, Queens, NY (US)

Correspondence Address: Yaguang Liu, 67-08 168th Street, Flushing, NY, 11365, US

(21) Appl. No.: 10/390,581

(22) Filed: Mar. 14, 2003

(51) Int. Cl. A61K 31/715; A61K 31/737; A61K 31/55; A61K 31/4745

(52) U.S. Cl. 514/54; 514/214.03; 514/283; 536/54

(57) ABSTRACT

The present invention relates to a new technique formed from polysaccharides of kelp (PK), which has function of anticancer and increasing immunity, and new nanoparticles (NP) and special liposome (SSL), which contained natural anticancer drug their preparation. Also, the present invention is aimed at the overall improvement of therapeutic efficacy of anticancer drugs, including Homoharringtonine (HHT), Curcumol (CUR), Eelemene (ELE) and Camptothecin (CPT) by NP and SSL. NP improves the anticancer therapeutic efficacy of HHT, CUR, ELE and CPT by using PK as polymer. PK can improve the anticancer therapeutic index and decrease side effect of free anticancer drugs. Also, PK has the function of increasing immunity.

The present invention disclosed a process for making a polysaccharide of kelp (PK), PK-Drug-NP and special PK-anticancer drug-containing sterically stabilized liposomes (PK-Drug-SSL).

### BACKGROUND OF THE INVENTION

The present invention relates to a new technique formed from natural anticancer polysaccharides of kelp (PK), which has function of anticancer and increasing immunity, and new nanoparticles (NP) which contained natural anticancer drug their preparation. Also, the present invention is aimed at the overall improvement of therapeutic efficacy of anticancer drugs including Homoharringtonine (HHT), Curcumol (CUR), Eelemene (ELE) and Camptothecin (CPT) by Nanoparticles (NP). NP improves the anticancer therapeutic efficacy of HHT, CUR, ELE and CPT by using PK as polymer. PK can improve the anticancer therapeutic index and decrease side effect of free anticancer drugs. Also, PK has the function of increasing immunity.

The present invention disclosed a process for making a polysaccharide of kelp (PK), PK-drug-NP and special PK-anticancer drug-containing sterically stabilized liposomes (PK-Drug-SSL).

### DESCRIPTION OF PRIOR ART

Pharmaceutical technology has grown and diversified rapidly in recent years. Controlled drug-delivery technology represents one of the frontier areas of science, which involves multidisciplinary scientific approach. The present invention disclosed new drug delivery systems. The new drug delivery system has many advantages, including improved efficacy, reduced toxicity, and improved patient compliance and convenience compared with conventional dosage forms.

Meanwhile, increasing for diseased cells and tissue by combination with a suitable drug carrier is a topic of interest in pharmaceutical research.

The use of NP as carrier for delivering drugs is important in cancer chemotherapy and intracellular anti-biotherapy.

Previous drug carriers have many problems, for example, liposomes are conventional carriers, which have been extensively studied. However, many technical factors have limited the development of liposomes in medicine: low efficiency of drug entrapment, rapid leakage of watersoluble drugs, poor storage stability and methods of preparation cannot use for large-scale production.

In addition, general NA has the risk of chronic toxicity due non-degraded polymer. Large-scale manufacture of these NA has never demonstrated. More important fact is the predominant uptake of NP, as well as other colloidal carriers, by phagocytic cells of the reticuloendothelial system (RES) located mainly in the liver and spleen and a resulting rapid clearance from the circulation have been a major obstacle to the delivery of drugs by NP to cells, tissues, or organs other than RES. Therefore, it is necessary to develop special NA by novel nanotechnology. Novel nanotechnology is an engineering discipline in which the goal to build devices and structures that have safe polymers for human being in the proper place.

So far, no any polysaccharides, which have anticancer and increasing immune functions, have been used as polymer for preparation of NP, which used for carrying of anticancer drugs. The present invention discloses that special polysaccharides, which have function of anticancer and increasing immune function, have been used as polymer for NP.

Further on, new NP and special liposome used as anticancer drug-delivery.

### DETAILED DESCRIPTION OF THE INVENTION

The present invention relates to a new technique formed from PK, new NP and special liposome, which contained natural anticancer drug their preparation. Also, the present invention is aimed at the overall improvement of therapeutic efficacy of anticancer drugs, including HHT, CUR, ELE and CPT by new NP. NP improves the therapeutic efficacy of HHT, CUR, ELE and CPT by using PK as polymer. PK is an ingredient of choice in the improving treatment of leukemia and solid tumors. In the case of cancer chemotherapy, PK can improve the therapeutic index and decrease side effect of anticancer drugs. Also, PK has the function of increasing immunity. Meanwhile, NP improves the therapeutic efficacy of HHT, CUR, ELE and CPT, by delayed clearance from the circulation, protecting anticancer drugs from biological environment. HHT, CUR, ELE and CPT in NP can decrease the uptake of anticancer drugs by normal tissue. Also, PK-Drug-NP, reduce the side effects of HHT, CUR, ELE and CPT.

Kelp is a food from several species of sea plants, including Laminaria japonica Aresch, Zostera marina L. or Phyllospadix scouleri Hook. The sea plants are indigenous to Yellow Sea and East Sea of china and it has been used as a Chinese and Japanese food for more than thousand years. It is also a food in the US food market. Therefore, kelp is very safe for human being.

PK is a desirable drug carrier, which has the following characters: nontoxic, biodegradable, biocompatible, and decomposed rapidly from the body. More important, PK has anticancer function and increases immune function. The special characters of PK plus advantage of PK-NP make a new preparation of anticancer drugs. These new preparations of anticancer drugs are PK-anticancer drug-NP and PK-anticancer drug-SSL. PK-anticancer drug-NP includes PK-HHT-NP, PK-CUR-NP, PK-ELE-NP and PK-CPT-NP. PK-anticancer drug-SSA includes PK-HHT-SSL, PK-CUR-SSL, PK-ELE-SSL and PK-CPT-SSL.

PK-HHT-NP and PK-HHT-SSL have stronger therapeutic efficacy of anticancer and lower side effect than free HHT. Other PK-Drug-NP and PK-Drug-SSL have same characters.

The present invention disclosed that a novel technique for the preparation of new PK-NP. This new KP-NP also exhibited obviously in clinics because increased blood circulation time and reduced organs accumulation. By contraries, general NP is difficult to be used because NP has been eliminated by reticuloendothelial system.

As mentioned above that many technical factors have limited the development of liposomes in medicine: low efficiency of drug entrapment, rapid leakage of watersoluble drugs, poor storage stability and methods of preparation cannot use for large-scale production. Additional, liposomes have been shown to interact preferentially with phagocytic reticuloendothelial cells, resulting in a high uptake of liposomes and their contents by the liver and spleen.

The present invention indicated that PK could improve liposome-delivered drug. PK-Drug-SSL could increase anticancer effects and decrease side effects of anticancer drug. Also, PK-Drug-SSL is stable in storage.

The following specific examples will provide detailed illustrations of methods of producing relative drugs, according to the present invention and pharmaceutical dosage units containing demonstrates its effectiveness in regulation of genes of cancer cells. These examples are not intended, however, to limit or restrict the scope of the invention in any way, and should not be construed as providing conditions, parameters, reagents, or starting materials which must be utilized exclusively in order to practice the present invention.

### EXAMPLE 1

### Preparation of the Polysaccharide

Finely powdered kelp was extracted with ether and with 80% ethanol in order to remove soluble components and residue obtained. The residue was further extracted with distilled water on a water-bath (80° C.). The hot extract was filtered and 95% of ethanol was added to filtrate and precipitates formed. The precipitates were collected by centrifugation, washed thoroughly with EtOH and ether, and dried. A grayish white powder was obtained. The dried powder was frozen overnight and allowed to thaw at room temperature. By the repeated freezing and thawing procedure, the powder was extracted by a cold water and removed soluble components. The resulting residue was chromatographed on DEAE-cellulose column. The column was eluted with hot water. The elution was concentrated by evaporation. The residue obtained and residue was freeze-dried. The final product is polysaccharide of kelp (KP).

The molecular weight is about 6000. The primary structure is β-(1-3)-G.

### EXAMPLE 2

### Anticancer Character of PK

The PK has been demonstrated a strong activity against sarcoma-180 and L-1210, L-615 (dosage is 20 mg/kg/day and inhibitor).

Material and Methods

Animals: Adult DBA/2J male mice, 6 to 8 weeks old. All animals weighed approximately 25 g when used in experiments. Mice were assigned randomly to treatment and control groups. Each member of which received identical dosed (i.p.) of PK or 0.9% NaCl solution for all injections. Volumes were 0.01 ml/g body weight.

Tumor: L1210 or P388 leukemia cells induced in mice by inoculation with L1210 or P388 leukemia cells (1.0×10⁵).

The leukemia cells were passed i.p. weekly. L1210 or P388 cells were cultured at 37° C. in RPMI-1640 medium (GIBCO) without antibiotics and supplemented with 10% fetal calf serum.

After the tumor implantation (1×10⁵ leukemia cells), PK was injected intraperitoneally once a day. The treatment was started between the end of 2nd and 6th day after inoculation. Dose of PK was 25 mg/kg/day for PK groups. Control mice were treated with same value of 0-9% NaCl. Anti-leukemia activity was expressed as T/C %. T is median survival days of treated group and C is that of control group. A T/C value was greater that 100% means the treated mice is surviving longer than the control mice. Those rats that survived 60 days after the last day of treatment were considered cured. The statistical significance of data was determined by student's test.

Test data are reported in Table 1-3. PK exhibits significant anti-leukemia against the experimental L1210 and P 388 in mice.

TABLE 1
Activity of PK against lymphoid leukemia a L1210

| Dose of PK (mg/kg/day) | Survival time (days) |
|---|---|
| Untreated | 8.9 ± 0.3 |
| PK | 12.5 ± 0.6* |

*P < 0.01: compared between treatment and control group;

TABLE 2
Activity of PK against P388 lymphocytic leukemia

| Dose of PK | |
|---|---|
| (mg/kg/day) | Survival time (days) |
| Untreated | 9.2 ± 1.1 |
| PK | 15.8 ± 2.5 |

TABLE 3
Effect of PK on sarcoma-180 solid tumors in mice*

| Dose | Number of mice | Mean survival (%)* |
|---|---|---|
| Control | 20 | 5 |
| PK | 20 | 35.0 |

*Percentage survival: 30 days observation after tumor cell inoculation.

The data of table 1-3 indicated that PK inhibits leukemia and solid tumors.

### EXAMPLE 3

### Effects of PK on Hemopoietic System

Effects of PK on hemopoietic system were investigated. Results showed that PK could markedly improve the recovery rate of hemopoieses in treatment mice by cyclophosphamide (CY).

The level of serum colony stimulating factor (CSF) increased after treatment of PK.

Pharmacological effects as illustrated as the following table.

TABLE 4

| Group | Number of sample | Mean (CFU-S ± SD) |
|---|---|---|
| Control | 10 | 32.20 ± 3.0 |
| CY | 10 | 7.6 ± 1.1 |
| PK + CY | 10 | 15.8 ± 2.0 |

The data of Table 4 indicated that PK protected the stem cells of bone marrow in mice from the killing effect of CY. PK also decreases the side effect of CY, which is an anticancer drug.

### EXAMPLE 4

### The Effect of PK on Phagocytosis of Peritoneal Macrophage and White Blood Cells

Male mice weight 18-20 g were used in the experiments and were divided into treated (PK) and control groups. The dosage of PK was 25 mg/kg injected intraperitoneally. The control mice were injected with same volume of normal saline. These injections were repeated daily for 5 days, both treated and control group were injected intraperitoneally with CY. The dosage of CY is 4.5 mg/kg.

Added 0.02 ml of 5% washed chick red blood cell suspension to 0.5 ml of the peritoneal exudates. Shook gently to mix and incubate at 37° C. for 5 minutes. Dipped two cover slips, closed to each other, in the above mixture and incubated for 30 minutes for the migration of the macrophages along the cover slips, fixed and stained with Sharma stain. Examined microscopically for:

Phagocytic rate—number of macrophages with phagocytized chick red blood cells per 100 macrophages counted. Concentration of PK and CY is the same as Example 2. Pharmacological effects are as illustrated by the following table.

TABLE 5

| | Number of | Phagocytic |
|---|---|---|
| Group | sample | (rate ± SD) |
| Control | 10 | 35.0 ± 4.8 |
| CY | 10 | 8.5 ± 0.90 |
| PK + CY | 10 | 12.8 ± 1.8 |

Action of PK and CY on white blood cells was investigated by means of white blood cells assay. PK protected white blood cells and phagocytosis from the killing effect of CY. The dosage of PK and CY is the same as in Example 4. Time of treatment is 10 days.

The results are listed below table:

TABLE 6

| | | Phagocytic |
|---|---|---|
| Group | Number of sample | (rate ± SD) |
| Control | 20 | 15.0 ± 1.7 |
| CY | 20 | 6.0 ± 0.70 |
| PK + CY | 20 | 9.2 ± 1.4 |

The data of Table 5-6 indicated that PK protected white blood cells and phagocytosis from the killing effect of CY.

### EXAMPLE 5

### The Effect of PK on Lymphoblastic Transformation

By means of ³H-TdR liquid scintillation assay technique, the action of PK on lymphoblastic transformation was investigated method:

(1) Experimental procedure of animal is the same as in Example 4.

(2) Lemphoblastic transformation test:

I. Reagents and Conditions for Cell Culture

a. Culture media—RPMI 1640, medium 199 minimal essential medium (Eagle).

b. Buffer—Hepes buffer, the final concentration at 37° C. was 25 mM, to maintain the pH of the medium at 7.31.

c. Serum—generally 15-205 fetal bovine serum was incorporated, for lymphocytes from mice, 5% was used.

d. Gaseous phase—5% CO₂ in air.

e. Cell concentration—generally 1-2 c 10⁶/ml.

f. Stimulants—20 μl/ml for phytohemagglutinin containing polysaccharide (PHA-M) or 10 μl/ml for polysaccharide-free purified phytohemagglutinin (PHA-P).

II. Measured by Liquid Scintillation

a. The conditions of cell culture are same as above. ³H-TdR was added after 48 hours of incubation at a final concentration of 1 μCi/ml and continued the incubation for 24 hours.

b. Washed the cells twice with cold normal saline and the erythrocytes were lysed. The intact lymphocytes were again washed once with cold saline. Spun down the lymphocytes and added 2 ml of 10% trichloroacetic acid to precipitate the protein. Washed twice with normal saline. Added 2 ml of ethanol:ether (1:1) to wash once. 0.2 ml of formic acid was then added for digestion till the precipitate was dissolved.

c. Added 4 ml of scintillation fluid to 0.1 ml of the final sample and counted in a liquid scintillation counter.

Results are listed in the following table: (concentration of PK and CY used is the same as that of Example 4.

TABLE 7

| Group | Number of sample | CPM ± SD |
|---|---|---|
| Normal | 10 | 1505 ± 130 |
| CY | 10 | 600 ± 65 |
| PK + CY | 10 | 905 ± 140 |

The data of Table 7 indicated that PK increased lymphoblastic transformation.

### EXAMPLE 6

### The Effect of PK on Interleukin-2 (rIL-2)

The method of determination rIL-2 was as same as Example 4. The experimental data are listed in the following table.

TABLE 8

| Group | Number of sample | IL-2 (U/ml) |
|---|---|---|
| Normal | 10 | 85.0 ± 9.0 |
| CY | 10 | 47.5 ± 5.5 |
| PK + CY | 10 | 60.2 ± 7.8 |

The data of Table 7 indicated that PK increased IL.

### EXAMPLE 7

### Effects of PK on Immune Function of Human Blood Lymphocytes

20 old volunteers (60-70 years of age) and 10 healthy young persons participated in the experiment.

2 ml of venous blood, heparinized was obtained from each of the participants. The Study of the effects of PK was carried out by using Eagle's Minimal Essential Medium MEM). MEM was supplemented with 0.125 ml of heat-inactivated fetal calf serum, 100 units of Penicillin and 0.1 mg of streptomycin per ml of medium. Culture medium was divided into treated (PK) and control group. PK was added to the culture medium of treatment group. The culture medium of control group was mixed with same volume as that of PK of normal saline on the 72 hours of culture. The ³H-thymidine (³H-TdR) was added into all the cultures (2 μci/ml) for last 12 hours of culture. The cells were harvested on 0.45 μm filters, washed with phosphate buffer (ph 7.4) and bleached with H₂O₂. The filters were then dried and the incorporation of ³H-TdR into lymphocytes cell was measured by scintillation counter.

TABLE 9

| | Young (n = 20) | | Old (n = 20) | |
|---|---|---|---|---|
| Index | Control | PK | Control | PK |
| CPM | 8305 ± 900 | 8385 ± 216 | 747 ± 95 | 1495 ± 156 |

| | | |
|---|---|---|
| T/C(%) | 101 | 200 |
| P | <0.01 | <0.01 |

According to Table 9, PK is found to increase lymphoblastic transformation of the old and young persons. However, PK is found to add nothing to the young persons. In other words, PK can increase human immune function in immunosuppressive state.

### EXAMPLE 8

### Effects of PK on G-CSF and TNF-α

Blood cells of healthy volunteers (HV) and patients with leukemia (PL) cultured at same condition of Example 8. 1 ml of PK (200 μ/ml) added to each well of a 48-hole culture plate. PK was added to each well. The culture plate was incubated for 16 hours in the incubator which contained 5% CO₂ at 37° C. The supernatants were collected for analysis. G-CSF levels were measured using the ELISA system of Quantikin R & D; TNF-α levels in the collected supernatant were measured using the ELISA system. Experiment was compared the differenced of G-CSF and TNF-α between healthy volunteers and patients with leukemia after treatment of PK.

TABLE 10
Effects of PK on G-CSF and TNF-α levels

| | |
|---|---|
| G-CSF | TNF-α |

| Group | HV | PL | HV | PL |
|---|---|---|---|---|
| Control | 14.0 ± 2.0 | 24.8 ± 5.5 | 18.5 ± 2.5 | 24.3 ± 12.5 |
| PK | 20.0 ± 2.8 | 36.8 ± 5.8 | 21.0 ± 2.8 | 32.2 ± 4.5 |

The data of Table 10 indicate that PK can increase G-CSF and TNF-α of healthy volunteers (HV) and patients with leukemia (PL). PK, therefore, is an effective agent used in increasing immune function for patients with cancer.

### EXAMPLE 9

### HHT Extraction

HHT was extracted from the skins, stems, leaves and seeds of Cephalotaxus fortunei Hook and other related species, such as Cephalotaxus sinensis Li, C. hainanensis, and C. wilsoniana.

1 kg of ground Cephalotaxus fortunei Hook was extracted with 8 liters of 90% ethanol at room temperature for 24 hrs. Filtered the solution to yield a filtrate A and filtercake. Percolated the filtercake with ethanol and filter again to yield filtrate B. Combined A and B, and distilled under reduced pressure to recover ethanol and an aqueous residue. To this residue, added 2% HCl to adjust the pH to 2.5. Separated the solids from the solution by filtration to yield a filtrate C. Washed the solids once with 2% HCl and filtered to yield a filtrate D. Combined C and D and adjusted the pH to 9.5 by adding saturated sodium carbonate solution. Extracted the alkaline filtrate with chloroform and separated the chloroform layer from the aqueous layer. Repeated this extraction process five times. Combined all the chloroform extracts and distilled at reduced pressure to recover chloroform and alkaloid as a solid residue respectively. The solid alkaloid was then dissolved in 6% citric acid in water. The solution adjusted to pH 7 and extracted with chloroform. The chloroform was concentrated under reduced pressure and then extracted with buffer of pH 6.7 and separated the layer of chloroform from buffer of pH 6.7. The chloroform was extracted with buffer of pH 5 and separated the layer of buffer of pH 5 from chloroform. The buffer was adjusted to pH 9 then extracted with chloroform. The chloroform was evaporated under reduced pressure and residue obtained. The residue chromatographed on column packed with alumina. The elution was chloroform. Elution of chloroform was chromatographed on silica gel. The silica gel packed column by wet method with litter chloroform. Elution was chloroform-pH 5 buffer. Elution was distilled under reduced pressure and residue obtained. Residue was purified by crystallization in methyl alcohol. Crystal was recrystallized in methyl alcohol. The purity of HHT was 99% as determined by HPLC and thin-layer chromatography.

It is important that producing PK-HHT-NP needs very pure HHT. The purity of 99% of HHT is satisfied for the requirement of making PK-HHT-NP. HHT has the following physical data:

Yield: 0.02%.

Melting point: 144°-146° C.

Infrared spectrum: 3500, 3400, 1665, 1030 and 940 cm-.

Ultraviolet spectrum: λpeak alcohol mμ (log γ): 240 (3.55), 290 (3.61).

### EXAMPLE 10

### Preparation of PK-HHT-NP (Method 1)

30 g of PK, 1 g of HHT and 10 g of poly lacticacid were dissolved in the mixed organic solvent of acetone (1 L) and dichloromethane (0.5 L). The resultant organic solution was emulsified into nanodroplets in 2 L of aqueous poly vinylalcohol (PVA) solution (2.0% w/v) under stirring at 15000 rpm using a homogenizer. Emulsified was evaporation under reduced pressure and organic solvents were removed. The dispersed NP solidified in the aqueous solution. The whole dispersed system was filtered with membrane filter (pore size: 1.0 μm) to separate NP. The NP dispersed in the filtrate was sedimentated by ultracentrifugation (150000 g×1 h) and recovered by removing the water. The sediment washed twice with ether and then dried at room temperature.

### EXAMPLE 11

### Preparation of PK-HHT-NP (Methods 2)

1 g of HHT and 300 g of PK was added to 1 liter of 1% dextran solution which containing 10 g/L of glucose (pH=3). Dextran solution was added to 10 L of corn oil or cottonseed oil and polylysine and then homogenized at 170° C. The resulting emulsion was then cooled to room temperature and the particles were precipitated by additional ether. The resulting particles were separated by centrifugation at 10,000 g for 20 minutes and washed twice with additional ether and then dried at room temperature (25° C.). The present NP can be expected to be used as carriers of various drugs and physiologically active agents, e.g., hormone, peptide and antigen, because of their biodegradability, and to be distributed into the systemic circulation after parenteral (intramuscular, subcutaneous, intravenous) administration. The NP was found to be very stable at a storage temperature of 4° C. for duration of six months.

It is very important that HHT is easily entrapped into nanoparticles at higher percentage relative to the loaded HHT.

The PK, corn oil and polylysine are safe natural products. Corn oil and polylysine also are eliminated in process of precipitation and washed. Therefore, it is safe preparation of NP.

TABLE 11

| | | | | |
|---|---|---|---|---|
| [symbol] Diameter (nm) | 74 | 80 | 85 | 95 |
| Particle size (%) | 10 | 50 | 38 | 2 |

Encapsulation efficiency of PK-HHT-NP is 87.9%. Drug recovery (%) was amount of drug in nanospheres/amount of drug fed in the system. Drug recovery (%) in PK-HHT-NP=8.5±7.6. Recovery of nanospheres (%)=96.6.

### EXAMPLE 12

### New Preparation of PK-HHT-NP (2)

HHT were dissolved in the mixed dextran (1% dextran in 0.01 NHCl) and glucose. Concentration of HHT was 5 g/L. Concentration of dextran and glucose was 10 g/L (pH=3.0). 10 g of poly butylcyanoacrylate (BCA) was added to 250 ml of HHT solution. Under stirring using homogenizer. Stirring time was 2 hours. The whole dispersed system was filtered with a membrane filter to separate NP. The NP dispersed in the filtrate was sedimentated by ultracentrifugation and recovered by removing the water. The sediment washed twice with ether and then dried at room temperature.

### EXAMPLE 13

### Preparation of PK-HHT-NP (3)

The inner water phase consisted of 10 g of HHT in a mixture containing 2.0 g of gelatin and water maintained at 60° C. The oil phase consisted of 100 g of PK and 40 G of poly lacticacid (PLA) and 2% of glyceryl monocaprate in 100 ml of dichloromethane. The oil phase was gradually poured into the inner water phase under vigorous stirring with homogenizer over a few minutes to make an emulsion. The emulsion was cooled to 15° C. and then poured into 8 L of a cooled 01% poly vinylalcohol solution under stirring. To evaporate dichloromethane, the emulsion was continuously stirred for two hours. The NP was collected by filtration and washed twice with additional ether and then dried at room temperature.

### EXAMPLE 14

### Preparation of PK-HHT-NP (4)

1 g of HHT and 150 g of PK was added to 1 liter of 5% human serum albumin solution and was homogenized for 10 minutes in 10 L of corn oil or cottonseed oil kept at 170-185° C. The resulting emulsion was then cooled to room temperature and the particles were precipitated by additional ether. The resulting particles were separated by centrifugation at 10,000 g for 20 minutes and washed twice with additional ether and then dried at room temperature (25° C.).

### EXAMPLE 15

### NP Purification

Nanocapsules were centrifuged at 55000×g for 2 hours. The supernatant was discarded and the pellet redispersed in double distilled water by mechanical stirring. NP was collected by filtration and washed twice with additional ether and then dried at room temperature.

### EXAMPLE 16

### CUR Extraction

CUR extracted from plant named Dryobalanops aromatica Gaerin or Wen E Shu. One kg of plant powder was extracted 5 L of water at room temperature for 12 hours. The powder was recovered by filtration. Filtrate A was saved and the powder filter residue was extracted with 4 L of water at room temperature for 10 hours. The mixture was filtered. Filtrate B was saved and powder filter residue was extracted for 3 L of water at room temperature for 8 hours. The mixture was filtered and filtrate C was saved. Filtrate A, B, and C was combined at distilled under 0.4 kg pressure for 32 hours. The oil and water fraction of distilled mixture was separated. The oil fraction was saved and kept temperature at 0° C. The crystals formed from oil fraction then crystals washed with petroleum ether. The needles crystal obtained after recrystallization from ethanol. The needle-crystal washed with petroleum ether and dried. The needle crystal is Curcumol. The experimental data of Curcumol are listed as the following.

Molecule form: C₁₅H₂₄O₂;

Molecule weight: 236.34

Colorness needle-crystal (from CH₃CH₂OH)

Mp: 141° C.˜142° C.

[α]²⁵: −40.8° (CH₃Cl)

[α]²⁵: −40.5° (CH₃CH₂OH)

Irν^{KBr} cm⁻¹: 3420 (—OH), 3096, 1645, 882 (CH₂═C<), 2962, 2872 (—CH₃), 2926, 2853 (—CH₂—), 1125 (—C—O—C); NMR (CDCl₃) δ: 0.89 (3H, d, CH₃, —CH<), 2.75 (2H, t, H₂C═C—CH2—(COH)—O—), 3.05 (1H, s, —OH), 4.80 (2H, t, H₂═C—CH₂—).

[image]

[image]

### EXAMPLE 17

### ELE Extraction

ELE used for treatment and prevention of malignant pleural effusion. One kg of plant powder was extracted 5 L of water at room temperature for 12 hours. The powder of plant named Dryobalanops aromatica Gaerin or Wen E Shu was recovered by filtration. Filtrate A was saved and the powder filter residue was extracted with 4 L of water at room temperature for 10 hours. The mixture was filtered. Filtrate B was saved and powder filter residue was extracted with 3 L of water at room temperature for 8 hours. The mixture was filtered and filtrate C was saved. Filtrate A, B, and C was combined at distilled under reduced pressure. The distilled mixture was separated. The oil fraction was saved and kept temperature at 0° C. The oil distilled under reduced pressure, (50°-80° C./40 Pa) and distillate (fraction A) was collected. Fraction A distilled under reduced pressure (76°-78° C./40 Pa) and Distillate (fraction B) was collected. Fraction B was then chromatographed on silica gel G, using petroleum ether as developing solvent. The solvent collected and dried. The final product is ELE. ELE has the following chemistry data.

Molecular form: C₁₅H₂₄

Molecular weight: 204

Mp: 114˜118° C.

[α]¹⁶: −15°

IRν^{KBr} cm⁻¹: 3090, 2975, 2860, 1642, 1440, 1375, 1002, 910, 888 (C═CH₂); PMR (CCl₄) δ: 0.97 (3H, s), 1.7 (6H, s), 4.4˜5.6 (6H, m), 5.7 (1H, dd, ═OH₂); MSm/e (%): 204 (M^{+),} 147 (33), 121 (41), 107 (54), 93 (89), 81 (100), 79 (44), 68 (74), 67 (52), 55 (41), 53 (33), 41(52).

### EXAMPLE 18

### Preparation of Curcumol-polysaccharides of Kelp-nanoparticles (PK-CUR-NP)

PK and poly lacticacid (PLA) was prepared as biodegradable polymeric carrier. Biodegradability of the polymer can avoid chromic toxicity of non-biodegradable polymer.

The 1.2 g of CUR, 20 g of PK and 10 g of PLA dissolved in 2.5 L of acetone-1.5 L of dichloromethane mixture was poured into 5 L of aqueous solution of polyvinyl alcohol with stirring (15000 rpm) using a high-speed homogenizer.

During evaporation of the water-immiscible organic solvent (dichloromethane or chloroform) from the droplets of mixed organic solution (for 3-4 h, the dispersed nanodroplets solidified in the aqueous solution. The whole dispersed system was filtered with a membrane filter. Filtrate was sedimentated by ultracentrifugation (156 200 g×1 h) and recovered by removing the water. Purification of PK-CUR-NP is same of Example 15. Additional, PLA is easily resolved in body. Lactic acid is natural and physical material. PK-CUR-NP preparation is safe.

### EXAMPLE 19

### Preparation of Polysaccharides of Kelp-elemene-nanoparticles (PK-ELE-NP)

1.2 g of ELE, 20 g of PK and 10 g of PLA dissolved in 2.5 L of acetone-1.5 L of dichloromethane mixture were poured into 5 L of aqueous solution of polyvinyl alcohol with stirring (15000 rpm) using a high-speed homogenizer.

During evaporation of the water-immiscible organic solvent (dichloromethane or chloroform) from the droplets of mixed organic solution (for 3-4 h), the dispersed nanodroplets solidified in the aqueous solution. The whole dispersed system was filtered with a membrane filter. Filtrate was sedimentated by ultracentrifugation (156 200 g×1 h) and recovered by removing the water. Purification of PK-ELE-NP is same of Example 15.

### EXAMPLE 20

### Camptothecine Extraction

1 kg of ground Camptoeca acuminata Decne was extracted with 8 liter of 95% ethanol at 50° C. for 24 hours. Filtered the solution to yield filter residue. The filter residue was extracted with 4 liter of CHCl₃. Filtered and yield filtrate. The filtrate was distilled under reduced pressure to recover CHCl₃ and obtained distilled residue. The distilled residue was extracted with methylic alcohol. Extraction of methylic alcohol was distilled under reduced pressure to recover methylic alcohol. The distilled residue was extracted by petroleum ether. Filtered the solution to yield filter residue. 10% NaOH added to filter residue under stirring. Solution of NaCH was warm by water bath and solution was filtered at 60° C. 2N HCl and methylic alcohol was added to solution of NaOH at 60° C. and sediment was obtained. Sediment was crystallized by CHCl₃—CH₃OH. The final product is camptothecine (CPT).

### EXAMPLE 21

### Preparation of Polysaccharides of Kelp-camptothecine-nanoparticles (PK-CPT-NP)

1.2 g of CAM, 20 g of PK and 10 g of PLA dissolved in 2.5 L of acetone-1.5 L of dichloromethane mixture were poured into 5 L of aqueous solution of polyvinyl alcohol with stirring (15000 rpm) using a high-speed homogenizer.

During evaporation of the water-immiscible organic solvent (dichloromethane or chloroform) from the droplets of mixed organic solution (for 3-4 h), the dispersed nanodroplets solidified in the aqueous solution. The whole dispersed system was filtered with a membrane filter. Filtrate was sedimentated by ultracentrifugation (156 200 g×1 h) and recovered by removing the water. The resulting particles washed twice with ether and dried at room temperature.

### EXAMPLE 22

### Radiolabelled Nanoparticle Experiment

Mice were used in this experiment. After administration of radiolabelled ³H-HHT or PK-³H-HHT-NP, 3 mice were killed at various time intervals, (0.5, 4.0 and 24.0 h).

After injection of radioactive ³H-HHT or PK-³H-HHT-NP, blood-associated radioactivity was determined.

1 ml of sample of blood was ultracentrifuged at 100,000×g for 1 hour. After discarding the supernatant, the sediment was dispersed again by mechanical stirring in 1.5 ml of distilled water. 100 μl of the sediment or supernatant were then added to 10 ml of scintillation liquid solution. After weighing, the radioactivity of the tissue samples was measured by a scintillation counter.

TABLE 12
Circulation ³H-HHT in blood

cpm

| Time after administration | PK-³H-HHT-NP | ³H-HHT (free) |
|---|---|---|
| 1 hour | 1980 | 2400 |
| 8 hours | 1300 | 90 |
| 24 hours | 1130 | 60 |

The data of Table 12 indicated that PK-HHT-NP dramatically prolonged elevation in HHT levels in blood compared to free HHT. Also, the data of Table 12 indicated that PK-HHT-NP is to enhance the therapeutic effect by creating a higher blood concentration of HHT at long time. It is more important in treatment of leukemia because leukemia is a blood cancer.

### EXAMPLE 23

### Anticancer Activity of PK-HHT-NP

The anticancer activity of PK-HHT-NP was compared in vitro and in vivo with that of free HHT treating leukemia and solid tumors. It was found that PK-HHT-NP showed higher anticancer activity. After treatment of cancer bearing mice with PK-HHT-NP, the survival rate was increased. This was due to the decreasing of side effects of HHT and increasing of anticancer activity by NP.

A. In vitro Test Methods

Human leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml.

Cell lines were routinely cultured in the RPMI1640 medium supplemented 20% fetal calf serum. The experiment was carried out in 96 microplate, each well had 5×10⁵ cells and given desired concentration of drug (1 μg/ml). Then the plate was incubated at 37° C. in an atmosphere of humidified air enriched with 5 percent carbon dioxide for 72 hours. Inhibition percent rate of tumor cell proliferation was obtained according to the bellow formula.

$$Inhibition percent rate = \frac{Control - Test}{Control} \times 100 \%$$

The results are summarized in the tables as below.

TABLE 13
Comparing anticancer activity of PK-HHT-NP and
free drug of HHT

| Treatment | Inhibition (%) |
|---|---|
| Control | 0 |
| HHT | 70.5 ± 8.0 |
| PK-HHT-NP* | 89.6 ± 9.5 |

*Dose of HHT-KP-NP is equivalent of free HHT.

B. In vivo Test

Methods: L-120 leukemia mice, weight 20-22 g were used in the experiment. The dose per injection was 5 mg HHT/hg body weight. The dose of PK-HHT-NP is equivalent of free HHT. (All the following comparing experiments are used same methods).

TABLE 14
Effects of free HHT and PK-HHT-NP on survival of
mice bearing leukemia

| Treatment | Dose mg/kg | Survival time T/L days | T/C (%) |
|---|---|---|---|
| Control | 0 | 9 | — |
| Free HHT | 1 | 5/9 | 55.6 |
| Free HHT | 5 | 27/9 | 300 |
| PK-HHT-NP | 1 | 34/9 | 378 |
| PK-HHT-NP | 5 | 40/9 | 444 |

The administration of PK-HHT-NP showed significantly increased survival time. These results showed that PK-HHT-NP caused a marked improvement in therapeutic efficiency, increased survival time.

### EXAMPLE 24

### The Effect of HHT and PK-HHT-NP on Decreasing of Tyrosine Kinase (TK)

In general, very low levels of TK are expressed in normal cells and high levels of TK are expressed in cancer cells. Many evidences have been accumulated that the dysfunction of cellular oncogenes is a cause of human cancers. Therefore, a drug, which inhibits the activity of TK, can provide a new way to overcome cancer.

Methods

Cells. L1210 and P388 cells were grown at 37° C. on medium RPMI-1640 without antibiotics and supplemented with 10% horse serum. Cultures were diluted daily to 1×10⁵ cells/ml with fresh growth medium. From a culture initiated with cells from ascitic fluid obtained from a mouse 5 days after implantation with in vivo-passage leukemia, a stock of ampoule containing 10⁷ cells/ml in growth medium plus 10% dimethyl sulfoxide was frozen and stored in liquid nitrogen. Cultures were started from the frozen stock and were passage for no more than 1 month.

L1210 and P388 cells were grown at 37° C. on medium RPMI-1640 supplemented with 10% calf serum. 10,000 unit/ml of Penicillin and 10,000 unit/ml of Streptomycin 1×10⁶/ml cells were placed in culture with different concentrations of HHT. Then the cell suspension was incubated at 37° C. in a humidified atmosphere of 5% CO₂-95% air for the indicated time. Reactions were terminated by addition of 3 ml of cold Earle's buffer. Cells were lysed, precipitated with 10% trichloroacetic acid (TCA) and filtered onto glass fiber filters. The filters were washed with phosphate-buffered saline and placed in scintillation vials, and radioactive emissions were counted.

H-60 leukemia cells were plated at a density of 5×10⁵ cells in 60-nm dished, and divided control and treatments groups for incubation 24 hours at 37° C. with 5% CO₂. The cells were collected and washed twice with phosphate-buffered saline and re-suspended at density of 10⁶ cells/ml in 5 mM HEPEs buffer (pH 7.4). The cells were then re-suspended in 1 ml of buffer containing 5 mM HEPES (pH 7.6), 1 mM MgCl₂ and 1 mM EDTA, then placed on ice bath. The cell membrane was disrupted by ultra sound and centrifuged at 1000×g for 10 minutes. The supernatant was ultra centrifuged at 30,000×g for 30 minutes at 4° C. The pellet was re-suspended in 0.3 ml of buffer containing 25 mM HEOES, centrifuged at 12,000×g for 5 minutes. The resulting supernatant was used for TK assay. Content of protein was determined. 10 μg of protein placed in 20 mM HEPES (pH 7.6), 15 mM MgCl₂, 10 mM ZnCl2 and 5% (v/v) Nonidet P-40. After 5 minutes incubation at 25° C., the reaction was initiated by the addition of 25 μM [γ³²P] ATP (3 ci/mmol). After 10 minutes, the reaction was stopped by the addition of 20 mM cold ATP. 50 μl of the mixtures were spotted on glass microfiber filter discs and washed three times with cold trichloroacetic acid (TCA), contained 10 mM sodium pyrophosphate. Air dried. Radioactivity was determined by liquid scintillation spectrometry. The net TK activity was determined after correcting for endogenous TK activity.

TABLE 15
Effect of HHT and PK-HHT-NP on TK activity of
HL-60 leukemia cells

| Group | Concentration (M) | % of control activity |
|---|---|---|
| Control | — | 100 |
| Free HHT | 10⁻⁷ | 14.8 |
| Free HHT | 10⁻⁶ | 20.8 |
| PK-HHT-NP | 10⁻⁷ | 12.5 |
| PK-HHT-NP | 10⁻⁶ | 3.8 |

Value represents the mean of two experiments each done in duplicate; the range was less than 5% of the mean.

The present study clearly demonstrated that PK-HHT-NP and HHT significantly reduced in TK activity. Also PK-HHT-NP is better than HHT.

### EXAMPLE 25

### Effects of HHT and PK-HHT-NP on Tumor Cells Proliferation

Materials and Methods:

Human tumor cell lines: Hela leukemia HL-60, malignant melanocarcinoma B16, oral epidermoid carcinoma (KB), lung carcinoma (A549), breast carcinoma MCF-7, adenocarcinoma of stomach.

Animal tumor cell lines: Walker carcinoma, LLC-WRC-256, malignant melanoma (RMMI 1846), 3T3, and S-180 sarcoma (CCRF-180). All lines were routinely cultured in the RPMI1640 medium supplemented 20% fetal calf serum. The experiment was carried out in 96 microplate, each well had 5×10⁵ cells and given desired concentration of 1 μg/ml (1×10⁻⁶ g/ml) drug. Then the plate was incubated at 37° C. in an atmosphere of humidified air enriched with 5 percent carbon dioxide for 72 hours.

Inhibition percent rate of tumor cell proliferation was obtained according to the bellow formula.

$$Inhibition percent rate = \frac{Control - Test}{Control} \times 100 \%$$

Results:

TABLE 16
Effect of PK-HHT-NP and HHT on inhibiting growth cancer cells

Inhibition (%)

| Cell line | HHT | PK-HHT-NP |
|---|---|---|
| Control | — | — |
| Human cells | | |
| HL-60 | 70.8 ± 9.0 | 89.0 ± 9.1 |
| Hela | 75.6 ± 8.0 | 82.5 ± 9.6 |
| B16 | 73.8 ± 8.1 | 85.8 ± 10.8 |
| KB | 80.0 ± 8.5 | 92.6 ± 10.4 |
| MCF-7 | 78.5 ± 7.9 | 93.7 ± 8.9 |

PK-HHT-NP and HHT inhibited tumor cells growth significantly (Table 16). PK-HHT-NP is better than free HHT.

### EXAMPLE 26

### Effect of HHT and PK-HHT-NP on Apoptosis of Cancer Cells

Methods

Human leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml.

Apoptosis determined by the following process: Cell pellets containing 5×10⁶ cells were fixed with 2.5% glutaraldehyde in cacodylate buffer (pH 7.4), dehydrated through graded alcohol, and infiltrated with LX-112 epoxy resin. After overnight polymerization at 60° C. 1-μm sections were cut with glass knives using a LKB Nova microtome. The sections were stained with 1% toluidine blue and coverslipped. In addition, experimental examples were stained with May-Grunwald-giemsa stain for the demonstration of apoptosis.

DNA electrophoresis: Untreated and treated HL-60 cells collected by centrifugation, washed in phosphate buffered saline and re-suspended at a concentration of 5×10⁶ cells and 0.1% RNase A. The mixture was incubated at 37° C. for 30 min and then incubated for an additional 30 min at 37° C. with 1 ml protease K. Buffer was added and 25 μl of the tube content transferred to the Horizontal 1.5% agarose gel electrophoresis was performed at 2 V/cm. DNA in gels visualized under UV light after staining with ethidium Bromide (5 μg/ml).

DNA fragmentation assays: DNA cleavage was performed, quantitation of fractional solubilized DNA by diphenylamine assay and the percentage of cells harboring fragmented DNA determined by in labeling techniques. For the diphenylamine assay, 5×10⁶ cells were lysed in 0.5 mL lysis buffer (5 mmol/L Tris-HCl, 20 mmol/L DTA, and 0.5% Triton X-100, pH 8.0) at 4° C. Lysates were centrifuged (15,000 g) for separation of high molecular weight DNA (pellet) and DNA cleavage products (supernatant). DNA was precipitated with 0.5 N perchloric acid and quantitated using diphenylamine reagent. The cell cycle distribution was determined 4 hours after addition of drug and represents mean±SD of 5 independent experiments.

TABLE 17
Effect of PK-HHT-NP and HHT on apoptosis of cancer cells

| Drug | |
|---|---|
| Concentration (μm) | Apoptosis (%) |
| Control | 0 |
| Free HHT (20) | 68.3 ± 7.6 |
| HHT-PK-NP (20) | 90.8 ± 10.8 |

The data of table 17 indicated that PK-HHT-NP and HHT could significantly induce apoptosis and PK-HHT-NP is better than HHT.

### EXAMPLE 27

### The Effect of HHT and PK-HHT-NP on the Growth of Transplanted Tumor

Experimental Procedure:

Male mice, weight 20-22 g, were used in the experiment. 1×10⁷ tumor cells were injected to mouse and PK-HHT-NP injected intraperitoneally began second day. All mice were sacrificed on the 12th days, isolated the tumor and weighed and calculated the inhibition rate of tumor weight.

Results:

The effect of PK-HHT-NP and HHT on the growth of animal transplanted tumor as illustrated by the Table 18. PK-HHT-NP 20 mg/kg could inhibit the growth of S180, ECS, HCS, ARS, U-14 and L615 transplanted tumor.

TABLE 18
Inhibition rate (%) of transplanted tumor

| | Inhibition (%) | |
|---|---|---|
| Transplanted tumor | HHT (20 mg/kg) | PK-HHT-NP (20 mg/kg) |
| Control | — | — |
| L1210 | 87 ± 9 | 91.8 ± 10.2 |
| Lewis | 70 ± 6 | 82.6 ± 9.8 |
| S180 | 76 ± 8 | 85.7 ± 10.2 |
| Walker 256 | 83 ± 9 | 92.8 ± 12.6 |

The data of Table 18 indicated that HHT and PK-HHT-NP could significantly inhibit growth of tumor and PK-HHT-NP is better than HHT.

According to above experiments, PK-HHT-NP increased selectivity to leukemic cells and solid tumors without any loss of efficacy. PK-HHT-NP reduced HHT toxicity for the normal tissues, mainly for the bone marrow and cardiac muscles. PK-HHT-NP modified the treatment schedules, e.q. reduced the administered doses. Anti-leukemic and tumor activity of PK-HHT-NP was proved to enhance as compared to the free HHT.

### EXAMPLE 28

### Comparative Anticancer Therapeutic Index of PK-CUR-NP and Free CUR

The acceptable criterion for determining the anti-tumor efficacy of the free CUR and PK-CUR-NP are the determination of the life span prolongation.

The number of dead cells in the peritoneal fluid of mice treated with PK-CUR-NP increased life span when compared to free CUR. This result, tabulated in Table 19, substantiates the histopathological study was performed, that by the 12^{th} day of tumor S-180 inoculation, the PK-CUR-NP exhibited a significantly higher tumor cell necrosis.

The results are tabulated in Table 19-20.

TABLE 19
Comparative evaluation of PK-CUR-NP and free CUR

| | | Number of stained dead |
|---|---|---|
| | | tumor cells |
| Group | Number of sample | (1 × 10⁶ cells mL⁻¹) |
| Normal mice | 10 | — |
| Tumor control | 10 | 1.95 ± 1.2 |
| Free CUR | 10 | 5.36 ± 0.95 |
| PK-CUR-NP drug | 10 | 9.57 ± 0.83 |
| P | | <0.001 |

TABLE 20
Comparative survival time of tumor-bearing mice after
treatment with free drug and PK-CUR-NP

| | | Increase in life span |
|---|---|---|
| Group | MST (days) | T/C (%) |
| Tumor control | 23 ± 1.2 | — |
| Placeo | 23 ± 2 | — |
| Free CUR | 31 ± 1.9 | 134.78 |
| PK-CUR-NP | 46 ± 2.3 | 200.0 |
| P | <0.001 | |

The data of Table 19-20 indicated that PK-CUR-NP has higher anti-tumor effect than a free CUR.

### EXAMPLE 29

### PK-CUR-NP and CUR Induce Differentiation of Cancer Cells

Human promyelocytic leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml. Differentiation was induced in HL-60 cells by treatment with 0.1 μg/ml (10×10⁻⁷ g/ml) of CUR or PK-CUR-NP. Cell differentiation was measured by the ability of cells to reduce NBT.

TABLE 21
Induced differentiation of CUR on cancer cells

| Treatment | NBT %* |
|---|---|
| None | 0 |
| Free CUR | 68.5 ± 8.5 |
| PK-CUR-NP | 76.8 ± 8.3 |

*NBT represents the percentage of cells capable of reducing NBT.

Increasing NBT % means that cancer cells are differentiation. The results of Table 21 indicate that free CUR and PK-CUR-NP markedly induced differentiation of human cancer cells and effect of PK-CUR-NP is much better than free CUR.

### EXAMPLE 30

### Effects of PK-CUR-NP and Free CUR on Tumor Cells Proliferation

Human tumor cell lines: Hela leukemia HL-60, malignant melanocarcinoma B16, oral epidermoid carcinoma (KB), lung carcinoma (A549), breast carcinoma MCF-7, adenocarcinoma of stomach.

Animal tumor cell lines: Walker carcinoma, LLC-WRC-256, malignant melanoma (RMMI 1846), 3T3, and S-180 sarcoma (CCRF-180). All lines were routinely cultured in the RPMI1640 medium supplemented 20% fetal calf serum. The experiment was carried out in 96 microplate, each well had 5×10⁵ cells and given desired concentration of 1 μg/ml (1×10⁻⁶ g/ml) drug. Then the plate was incubated at 37° C. in an atmosphere of humidified air enriched with 5 percent carbon dioxide for 72 hours. Concentration of CUR or PK-CUR-NP is 50ng/ml.

Inhibition percent rate of tumor cell proliferation was obtained according to the bellow formula.

$$Inhibition percent rate = \frac{Control - Test}{Control} \times 100 \%$$

TABLE 22
Effect of PK-CUR-NP and free CUR on inhibiting
growth cancer cells

Inhibition (%)

| Cell line | Free CUR | PK-CUR-NP |
|---|---|---|
| Control | — | — |
| Human cells | | |
| HL-60 | 76.8 ± 12.8 | 84.0 ± 11.8 |
| Hela | 67.2 ± 10.4 | 79.8 ± 10.1 |
| KB | 60.8 ± 9.1 | 84.2 ± 9.8 |

The data of Table 22 indicated that PK-CUR-NP has higher anticancer effect than a free CUR.

### EXAMPLE 31

### Effect of PK-CUR-NP and Free CUR on Apoptosis of Cancer Cells

Human promyelocytic leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml. Apoptosis determined by the following process: Cell pellets containing 5×10⁶ cells ere fixed with 2.5% glutaraldehyde in cacodylate buffer (pH 7.4), dehydrated through graded alcohol, and infiltrated with LX-112 epoxy resin. After overnight polymerization at 60° C. 1-μm sections were cut with glass knives using a LKB Nova microtome. The sections were stained with 1% toluidine blue and coverslipped. In addition, experimental examples were stained with May-Grunwald-giemsa stain for the demonstration of apoptosis.

DNA electrophoresis: Untreated and treated HL-60 cells collected by centrifugation, washed in phosphate buffered saline and resuspended at a concentration of 5×10⁶ cells and 0.1% RNase A. The mixture was incubated at 37° C. for 30 min and then incubated for an additional 30 min at 37° C. with 1 ml protease K. Buffer was added and 25 μl of the tube content transferred to the Horizontal 1.5% agarose gel electrophoresis was performed at 2 V/cm. DNA in gels visualized under UV light after staining with ethidium Bromide (5 μg/ml).

DNA fragmentation assays: DNA cleavage was performed, quantitation of fractional solubilized DNA by diphenylamine assay and the percentage of cells harboring fragmented DNA determined by in labeling techniques. For the diphenylamine assay, 5×10⁶ cells were lysed in 0.5 mL lysis buffer (5 mmol/L Tris-HCl, 20 mmol/L DTA, and 0.5% Triton X-100, pH 8.0) at 4° C. Lysates were centrifuged (15,000 g) for separation of high molecular weight DNA (pellet) and DNA cleavage products (supernatant). DNA was precipitated with 0.5N perchloric acid and quantitated using diphenylamine reagent. The cell cycle distribution was determined 4 hours after addition of drug and represents mean±SD of 5 independent experiments.

TABLE 23
Effect of free CUR and PK-CUR-NP on apoptosis of cancer cells

| Drug | |
|---|---|
| Concentration (μg) | Apoptosis (%) |
| Control | 0 |
| CUR | 68.3 ± 7.8 |
| PK-CUR-NP | 84.2 ± 9.1 |

The data of Table 23 indicated that free CUR and PK-CUR-NP could significantly induce apoptosis. And PK-CUR-NP is better than free CUR.

### EXAMPLE 32

### The Effect of Free CUR and PK-CUR-NP on the Growth of Transplanted Tumor

Experimental Procedure:

Male mice, weight 20-22 g, were used in the experiment. 1×10⁷ tumor cells were injected to mouse and CUR and PK-CUR-NP injected intraperitoneally began second day. All mice were sacrificed on the 12th days, isolated the tumor and weighed and calculated the inhibition rate of tumor weight.

Results:

The effect of CUR and PK-CUR-NP on the growth of animal transplanted tumor as illustrated by the Table 24. CUR and PK-CUR-NP 20 mg/kg could inhibit the growth of S180, ECS, HCS, ARS, U-14 and L615 transplanted tumor.

TABLE 24
Inhibition rate (%) of transplanted tumor

Inhibition (%)

| Transplanted tumor | Free CUR | PK-CUR-NP |
|---|---|---|
| Control | — | — |
| L1210 | 70.8 ± 9.0 | 85.0 ± 9.3 |
| Lewis | 68.9 ± 7.8 | 80.9 ± 10.1 |
| S180 | 63.7 ± 7.3 | 89.7 ± 12.0 |
| Walker 256 | 59.8 ± 8.5 | 78.8 ± 8.5 |

The data of Table 24 indicated that free CUR and PK-CUR-NP could significantly inhibit growth of tumor. PK-CUR-NP is better than free CUR.

### EXAMPLE 33

### Free CUR and PK-CUR-NP Inhibited Tumor Incidence in vivo

The capacity of tobacco-specific nitrosamine 4-(methylnitrosamino)-1-(3-pyridyl)-1-butanone (NNK) to induce tumor incidence was recognized.

Methods:

Every group had 20 mice. For treatment group, each mouse was gave free CUR and PK-CUR-NP by injection at dose of 20 mg/kg daily. For control group, each mouse was gave same volume of physiological saline. Three days later, mice were gave 10 μmol NNK (in 0.1 ml saline) by i.p. injection. Sixteen weeks after these treatments the mice were killed and pulmonary adenomas were counted. The statistical significance of bioassay data was determined by student's test.

TABLE 25
Effects of free CUR and PK-CUR-NP on
NNK-induced lung tumorigenesis

| Group | Tumor incidence (%) | P |
|---|---|---|
| Control | 100 | — |
| Free CUR | 35.8 ± 5.8 | <0.01 |
| PK-CUR-NP | 28.9 ± 5.0 | <0.01 |

Data of Table 25 indicated that free CUR and PK-CUR-NP have a significant tumor incidence. However, PK-CUR-NP is better than free CUR.

According to above experiments, PK-CUR-NP increased selectivity to cancer cells without any loss of CUR efficacy. PK-CUR-NP reduced CUR toxicity for the normal tissues, mainly for the bone marrow and cardiac muscles. PK-CUR-NP modified the treatment schedules, e.q. and reduced the administered doses. Anticancer activity of PK-CUR-NP was proved to enhance as compared to the free CUR.

### EXAMPLE 34

### Comparative Anticancer Therapeutic Index of PK-ELE-NP and Free ELE

The acceptable criterion for determining the anti-tumor efficacy of the formulation over the free ELE and PK-ELE-NP are the determination of the life span prolongation.

The number of dead cells in the peritoneal fluid of mice treated with PK-ELE-NP formulation increased life span when compared to free ELE. This result, tabulated in Table 26-27, substantiates the histopathological study, that by the 12^{th} day of tumor inoculation, the PK-ELE-NP formulation exhibited a significantly higher tumor cell necrosis.

The results are tabulated in Table 26-27.

TABLE 26
Comparative evaluation of PK-ELE-NP and free ELE

| | | Number of stained dead |
|---|---|---|
| | | tumor cells |
| Group | Number of sample | (1 × 10⁶ cells mL⁻¹) |
| Normal mice | 10 | — |
| Tumor control | 10 | 1.95 ± 1.2 |
| Free ELE | 10 | 5.80 ± 0.80 |
| PK-ELE-NP | 10 | 9.90 ± 12.8 |
| P | | <0.001 |

TABLE 27
Comparative survival time of tumor-bearing mice after
treatment with free ELE and PK-ELE-NP

| | | Increase in life span |
|---|---|---|
| Group | MST (days) | T/C (%) |
| Tumor control | 23 ± 1.2 | — |
| Placeo | 23 ± 2 | — |
| Free ELE | 33.2 | 144.3 |
| PK-ELE-NP | 60.5 | 250.0 |
| P | <0.001 | |

The data of Table 26-27 indicated that the effect of PK-ELE-NP is better than free ELE.

### EXAMPLE 35

### PK-ELE-NP and ELE Induce Differentiation of Cancer Cells

Human promyelocytic leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml. Differentiation was induced in HL-60 cells by treatment with 0.1 μg/ml (10×10⁻⁷ g/ml) of ELE or PK-ELE-NP. Cell differentiation was measured by the ability of cells to reduce NBT. Increasing NBT % means that cancer cells are differentiation.

TABLE 28
Induced differentiation of free ELE and
PK-ELE-NP on cancer cells

| Treatment | NBT %* |
|---|---|
| None | 0 |
| Free ELE | 69.5 ± 10.1 |
| PK-ELE-NP | 81.2 ± 12.5 |

*NBT represents the percentage of cells capable of reducing NBT.

The results of Table 28 indicate that free ELE and PK-ELE-NP markedly induced differentiation of human cancer cells and effect of PK-ELE-NP is much better than free ELE.

### EXAMPLE 36

### Effects of PK-ELE-NP and Free ELE on Tumor Cells Proliferation

Human tumor cell lines: Hela leukemia HL-60, malignant melanocarcinoma B16, oral epidermoid carcinoma (KB), lung carcinoma (A549), breast carcinoma MCF-7, adenocarcinoma of stomach.

Animal tumor cell lines: Walker carcinoma, LLC-WRC-256, malignant melanoma (RMMI 1846), 3T3, and S-180 sarcoma (CCRF-180). All lines were routinely cultured in the RPMI1640 medium supplemented 20% fetal calf serum. The experiment was carried out in 96 microplate, each well had 5×10⁵ cells and given desired concentration of 1 μg/ml (1×10⁻⁶ g/ml) drug. Then the plate was incubated at 37° C. in an atmosphere of humidified air enriched with 5 percent carbon dioxide for 72 hours. Concentration of ELE or PK-ELE-NP is 50 ng/ml.

Inhibition percent rate of tumor cell proliferation was obtained according to the bellow formula.

$$Inhibition percent rate = \frac{Control - Test}{Control} \times 100 \%$$

ELE inhibited tumor cells growth significantly. Percent rates of inhibition were all more than 70% in all cancer cells by ELE.

TABLE 29
Effect of PK-ELE-NP and free ELE on
inhibiting growth cancer cells

Inhibition (%)

| Cell line | Free ELE | PK-ELE-NP |
|---|---|---|
| Control | — | — |
| Human cells | | |
| HL-60 | 78.9 ± 12.5 | 86.9 ± 7.8 |
| Hela | 69.8 ± 11.2 | 80.2 ± 11.2 |
| KB | 60.2 ± 13.2 | 85.8 ± 14.2 |

The data of Table 29 indicated that PK-ELE-NP has higher anticancer effect than a free ELE.

### EXAMPLE 37

### Effect of PK-ELE-NP and Free ELE on Apoptosis of Cancer Cells

Human promyelocytic leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml. Apoptosis determined by the following process: Cell pellets containing 5×10⁶ cells ere fixed with 2.5% glutaraldehyde in cacodylate buffer (pH 7.4), dehydrated through graded alcohol, and infiltrated with LX-112 epoxy resin. After overnight polymerization at 60° C. 1-μm sections were cut with glass knives using a LKB Nova microtome. The sections were stained with 1% toluidine blue and coverslipped. In addition, experimental examples were stained with May-Grunwald-giemsa stain for the demonstration of apoptosis.

DNA electrophoresis: Untreated and treated HL-60 cells collected by centrifugation, washed in phosphate buffered saline and resuspended at a concentration of 5×10⁶ cells and 0.1% RNase A. The mixture was incubated at 37° C. for 30 min and then incubated for an additional 30 min at 37° C. with 1 ml protease K. Buffer was added and 25 μl of the tube content transferred to the Horizontal 1.5% agarose gel electrophoresis was performed at 2 V/cm. DNA in gels visualized under UV light after staining with ethidium Bromide (5 μg/ml).

DNA fragmentation assays: DNA cleavage was performed, quantitation of fractional solubilized DNA by diphenylamine assay and the percentage of cells harboring fragmented DNA determined by in labeling techniques. For the diphenylamine assay, 5×10⁶ cells were lysed in 0.5 mL lysis buffer (5 mmol/L Tris-HCl, 20 mmol/L DTA, and 0.5% Triton X-100, pH 8.0) at 4° C. Lysates were centrifuged (15,000 g) for separation of high molecular weight DNA (pellet) and DNA cleavage products (supernatant). DNA was precipitated with 0.5N perchloric acid and quantitated using diphenylamine reagent. The cell cycle distribution was determined 4 hours after addition of drug and represents mean±SD of 5 independent experiments.

TABLE 30
Effect of free ELE and PK-ELE-NP on apoptosis
of cancer cells

| Drug | |
|---|---|
| Concentration (μg) | Apoptosis (%) |
| Control | 0 |
| Free ELE | 69.2 ± 9.1 |
| PK-ELE-NP | 86.8 ± 10.2 |

The data of Table 30 indicated that free ELE and PK-ELE-NP could significantly induce apoptosis. PK-ELE-NP is better than free ELE.

### EXAMPLE 38

### The Effect of Free ELE and PK-ELE-NP on the Growth Transplanted Tumor

Experimental Procedure:

Male mice, weight 20-22 g, were used in the experiment. 1×10⁷ tumor cells were injected to mouse and ELE injected intraperitoneally began second day. All mice were sacrificed on the 12th days, isolated the tumor and weighed and calculated the inhibition rate of tumor weight.

Results:

The effect of ELE on the growth of animal transplanted tumor as illustrated by the Table 30. ELE 20 mg/kg could inhibit the growth of S180, ECS, HCS, ARS, U-14 and L615 transplanted tumor.

TABLE 31
Inhibition rate (%) of transplanted tumor

Inhibition (%)

| Transplanted tumor | Free ELE | PK-ELE-NP |
|---|---|---|
| Control | — | — |
| L1210 | 71.8 ± 9.5 | 89.8 ± 9.8 |
| Lewis | 65.8 ± 10.5 | 78.9 ± 8.9 |
| S180 | 67.57 ± 9.1 | 88.5 ± 10.2 |
| Walker 256 | 60.8 ± 8.9 | 79.8 ± 10.5 |

The data of Table 31 indicated that free ELE and PK-ELE-NP could significantly inhibit growth of tumor. PK-ELE-NP is better than free ELE.

### EXAMPLE 39

### Free ELE and PK-ELE-NP Inhibited Tumor Incidence in vivo

The capacity of tobacco-specific nitrosamine 4-(methylnitrosamino)-1-(3-pyridyl)-1-butanone (NNK) to induce tumor incidence was recognized.

Methods:

Every group had 20 mice. For treatment group, each mouse was gave ELE and PK-ELE-NP by injection at dose of 20 mg/kg daily. For control group, each mouse was gave same volume of physiological saline. Three days later, mice were gave 10 μmol NNK (in 0.1 ml saline) by i.p. injection. Sixteen weeks after these treatments the mice were killed and pulmonary adenomas were counted. The statistical significance of bioassay data was determined by student's test.

TABLE 32
Effects of free ELE and PK-ELE-NP on NNK-induced
lung tumorigenesis

| Group | Tumor incidence (%) | P |
|---|---|---|
| Control | 100 | — |
| Free ELE | 34.8 ± 6.2 | <0.01* |
| PK-ELE-NP | 26.8 ± 8.3 | <0.01* |

*Compared with control group

The data of Table 32 indicated that free ELE and PK-ELE-NP have a significant tumor incidence. However, PK-ELE-NP is better than free ELE.

According to above experiments, PK-ELE-NP increased selectivity to cancer cells without any loss of efficacy. PK-ELE-NP reduced ELE toxicity for the normal tissues, mainly for the bone marrow. PK-ELE-NP modified the treatment schedules, e.q. and reduced the administered doses. PK-ELE-NP, anticancer activity was proved to enhance as compared to the free ELE.

### EXAMPLE 40

### Comparative Anticancer Therapeutic Index of PK-CPT-NP and Free CPT

The acceptable criterion for determining the anti-tumor efficacy of the formulation over the free drug and PK-CPT-NP are the determination of the life span prolongation. The number of dead cells in the peritoneal fluid of mice treated with PK-CPT-NP formulation increased life span when compared to free CPT. This result, tabulated in Table 33-34, substantiates the histopathological study, that by the 12^{th} day of tumor inoculation, the PK-CPT-NP exhibited a significantly higher tumor cell necrosis.

The results are tabulated in Table 33-34.

TABLE 33
Comparative evaluation of PK-CPT-NP and free CPT

| | | Number of stained dead |
|---|---|---|
| | | tumor cells |
| Group | Number of sample | (1 × 10⁶ cells mL⁻¹) |
| Normal mice | 10 | — |
| Tumor control | 10 | 1.95 ± 1.2 |
| Free CPT | 10 | 5.36 ± 0.95 |
| PK-CPT-NP | 10 | 9.57 ± 0.83 |
| P | | <0.001 |

TABLE 34
Comparative survival time of tumor-bearing mice after
treatment with free CPT and PK-CPT-NP

| | | Increase in life span |
|---|---|---|
| Group | MST (days) | T/C (%) |
| Tumor control | 23 ± 1.2 | — |
| Placeo | 23 ± 2 | — |
| Free CPT | 33 ± 4.5 | 144.3 |
| PK-CPT-NP | 68.9 ± 6.5 | 299 |
| P | <0.001 | |

The data of Table 33-34 indicated that PK-CPT-NP has higher anticancer effect than a free CPT.

### EXAMPLE 41

### PK-CPT-NP and CPT Controls Differentiation of Cancer Cells

Human promyelocytic leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml. Differentiation was induced in HL-60 cells by treatment with 0.1 μg/ml (10×10⁻⁷ g/ml) of CPT or PK-CPT-NP. Cell differentiation was measured by the ability of cells to reduce NBT. Increasing NBT % means that cancer cells are differentiation.

TABLE 35
Induced differentiation of free CPT and PK-CPT-NP
on cancer cells

| Treatment | NBT %* |
|---|---|
| None | 0 |
| Free CPT | 65.8 ± 9.1 |
| PK-CPT-NP | 79.5 ± 12.2 |

*NBT represents the percentage of cells capable of reducing NBT.

The results of Table 35 indicate that free CPT and PK-CPT-NP markedly induced differentiation of human cancer cells and effect of PK-CPT-NP is much better than free CPT.

### EXAMPLE 42

### Effects of PK-CPT-NP and Free CPT on Tumor Cells Proliferation

Human tumor cell lines: Hela leukemia HL-60, malignant melanocarcinoma B16, oral epidermoid carcinoma (KB), lung carcinoma (A549), breast carcinoma MCF-7, adenocarcinoma of stomach.

Animal tumor cell lines: Walker carcinoma, LLC-WRC-256, malignant melanoma (RMMI 1846), 3T3, and S-180 sarcoma (CCRF-180). All lines were routinely cultured in the RPMI1640 medium supplemented 20% fetal calf serum. The experiment was carried out in 96 microplate, each well had 5×10⁵ cells and given desired concentration of 1 μg/ml (1×10⁻⁶ g/ml) drug. Then the plate was incubated at 37° C. in an atmosphere of humidified air enriched with 5 percent carbon dioxide for 72 hours. Concentration of CPT or PK-CPT-NP is 50 ng/ml.

Inhibition percent rate of tumor cell proliferation was obtained according to the bellow formula.

$$Inhibition percent rate = \frac{Control - Test}{Control} \times 100 \%$$

CPT inhibited tumor cells growth significantly.

TABLE 36
Effect of PK-CPT-NP and free CPT on inhibiting
growth cancer cells

Inhibition (%)

| Cell line | Free CPT | PK-CPT-NP |
|---|---|---|
| Control | — | — |
| Human cells | | |
| HL-60 | 70.8 ± 11.2 | 82.8 ± 12.2 |
| Hela | 60.8 ± 12.2 | 78.8 ± 11.8 |
| KB | 598 ± 14.2 | 76.8 ± 12.0 |

The data of Table 36 indicated that the inhibiting effect of PK-CPT-NP is better than free CPT.

### EXAMPLE 43

### Effect of PK-CPT-NP and Free CPT on Apoptosis of Cancer Cells

Human promyelocytic leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml. Apoptosis determined by the following process: Cell pellets containing 5×10⁶ cells ere fixed with 2.5% glutaraldehyde in cacodylate buffer (pH 7.4), dehydrated through graded alcohol, and infiltrated with LX-112 epoxy resin. After overnight polymerization at 60° C. 1-μm sections were cut with glass knives using a LKB Nova microtome. The sections were stained with 1% toluidine blue and coverslipped. In addition, experimental examples were stained with May-Grunwald-giemsa stain for the demonstration of apoptosis.

DNA electrophoresis: Untreated and treated HL-60 cells collected by centrifugation, washed in phosphate buffered saline and resuspended at a concentration of 5×10⁶ cells and 0.1% RNase A. The mixture was incubated at 37° C. for 30 min and then incubated for an additional 30 min at 37° C. with 1 ml protease K. Buffer was added and 25 μl of the tube content transferred to the Horizontal 1.5% agarose gel electrophoresis was performed at 2 V/cm. DNA in gels visualized under UV light after staining with ethidium Bromide (5 μg/ml).

DNA fragmentation assays: DNA cleavage was performed, quantitation of fractional solubilized DNA by diphenylamine assay and the percentage of cells harboring fragmented DNA determined by in labeling techniques. For the diphenylamine assay, 5×10⁶ cells were lysed in 0.5 mL lysis buffer (5 mmol/L Tris-HCl, 20 mmol/L DTA, and 0.5% Triton X-100, pH 8.0) at 4° C. Lysates were centrifuged (15,000 g) for separation of high molecular weight DNA (pellet) and DNA cleavage products (supernatant). DNA was precipitated with 0.5N perchloric acid and quantitated using diphenylamine reagent. The cell cycle distribution was determined 4 hours after addition of drug and represents mean±SD of 5 independent experiments.

TABLE 37
Effect of CPT on apoptosis of cancer cells

| Drug | |
|---|---|
| Concentration (μg) | Apoptosis (%) |
| Control | 0 |
| CPT | 65.8 ± 9.8 |
| PK-CPT-NP | 82.8 ± 12.8 |

The data of Table 37 indicated that free CPT and PK-CPT-NP could significantly induce apoptosis. Also, PK-CPT-NP is better than free CPT.

### EXAMPLE 44

### The Effect of CPT on the Growth of Animal Transplanted Tumor

Experimental Procedure:

Male mice, weight 20-22 g, were used in the experiment. 1×10⁷ tumor cells were injected to mouse and CPT injected intraperitoneally began second day. All mice were sacrificed on the 12th days, isolated the tumor and weighed and calculated the inhibition rate of tumor weight.

Results:

The effect of CPT on the growth of animal transplanted tumor as illustrated by the Table 38. CPT 20 mg/kg could inhibit the growth of S180, ECS, HCS, ARS, U-14 and L615 transplanted tumor.

TABLE 38
Inhibition rate (%) of transplanted tumor

Inhibition (%)

| Transplanted tumor | Free CPT | PK-CPT-NP |
|---|---|---|
| Control | — | — |
| L1210 | 68.5 ± 9.8 | 87.2 ± 10.2 |
| Lewis | 60.8 ± 10.4 | 78.2 ± 12.1 |
| S180 | 63.2 ± 12.1 | 80.2 ± 11.2 |
| Walker 256 | 71.8 ± 10.4 | 89.9 ± 12.2 |

The data of Table 38 indicated that free CPT and PK-CPT-NP could significantly inhibit growth of tumor. PK-CPT-NP is better than free CPT.

### EXAMPLE 45

### Free CPT and PK-CPT-NP Inhibited Tumor Incidence in vivo

The capacity of tobacco-specific nitrosamine 4-(methylnitrosamino)-1-(3-pyridyl)-1-butanone (NNK) to induce tumor incidence was recognized.

Methods:

Every group had 20 mice. For treatment group, each mouse was gave free CPT and PK-CPT-NP by injection at dose of 20 mg/kg daily. For control group, each mouse was gave same volume of physiological saline. Three days later, mice were gave 10 μmol NNK (in 0.1 ml saline) by i.p. injection. Sixteen weeks after these treatments the mice were killed and pulmonary adenomas were counted. The statistical significance of bioassay data was determined by student's test.

TABLE 39
Effects of free CPT and PK-CPT-NP on
NNK-induced lung tumorigenesis

| Group | Tumor incidence (%) | P |
|---|---|---|
| Control | 100 | — |
| Free CPT | 36.2 ± 6.2 | <0.01 |
| PK-CPT-NP | 30.8 ± 7.2 | <0.01 |

The data of Table 39 indicated that free CPT and PK-CPT-NP have a significant tumor incidence, and PK-CPT-NP is better than free CPT.

According to above experiments, PK-CPT-NP increased selectivity to leukemic cells without any loss of CPT efficacy. PK-CPT-NP reduced CPT toxicity for the normal tissues, mainly for the bone marrow. PK-CPT-NP modified the treatment schedules, e.q. and reduced the administered doses. PK-CPT-NP, anticancer activity was proved to enhance as compared to the free CPT.

### EXAMPLE 46

### Safety of Composition of HHT

1. LD₅₀: The LD₅₀ of free HHT in mice (I.P.) was found to be 3.3±0.29 mg/kg.

2. LD₅₀ of PK-HHT-NP in mice (I.P.) was found to be 7.8±0.68 mg/kg.

3. Each dose for an adult was 50-500 mg. Using 50 kg as the average weight of an adult the dosage of PK-HHT-NP was 1-10 mg/kg, therefore, it was very safe.

According to above experiments, PK-HHT-NP increased safety without any loss of HHT efficacy. PK-HHT-NP reduced HHT toxicity for normal tissues, mainly for the bone marrow and cardiac muscles. PK-HHT-NP modified the treatment schedules, e.q. and reduced the administered doses.

Treating leukemia and tumor activity of PK-HHT-NP was proved to enhance as compared to the free HHT.

LD₅₀ of PK-HHT-NP is higher than LD₅₀ of free HHT. The above toxicology data mean that PK-HHT-NP is safer than free HHT.

### EXAMPLE 47

### Safety of Composition of CUR

1. The acute LD₅₀ of free CUR was found to be 325 mg/kg injection in abdominal cavity in mice. The acute LD₅₀ of PK-CUR-NP was found to be 780 mg/kg injection in abdominal cavity in mice.

2. Each dose for an adult was 50-500 mg. Using 50 kg as the average weight of an adult the dosage of PK-CUR-NP was 1-10 mg/kg, therefore, it was very safe.

3. As to subacute toxicity tests, a dosage corresponding to 50 times of the clinical dose of CUR was administered continually for two months, and no side effects had been observed. The electrocardiograms and functions of liver and the kidney had not been effect and no injuries whatever had been observed in the tissue slices of the heart, liver, spleen, lungs, kidneys and adrenal.

4. LD₅₀ of PK-CUR-NP is higher than CUR. It means that PK-CUR-NP is safer than free CUR.

### EXAMPLE 48

### Safety of Composition of ELE

1. The acute LD₅₀ of ELE was found to be 318 mg/kg injection in abdominal cavity in mice. LD₅₀ of PK-ELE-NP was 760 mg/kg.

2. Each dose for an adult was 50-500 mg. Using 50 kg as the average weight of an adult the dosage of PK-ELE-NP were 1-10 mg/kg, and they are very safe.

3. As to subacute toxicity tests, a dosage corresponding to 50 times the clinical dose of free ELE and PK-ELE-NP was administered continually for two months, and no side effects had been observed. The electrocardiograms and functions of liver and the kidney had not been effect and no injuries whatever had been observed in the tissue slices of the heart, liver, spleen, lungs, kidneys and adrenal.

4. LD₅₀ of PK-ELE-NP is higher than LD₅₀ of ELE. It means that PK-ELE-NP is safer than free ELE.

### EXAMPLE 49

### Safety of Composition of CPT

1. The acute LD₅₀ of CPT was found to be 76 mg/kg injection in abdominal cavity in mice. And LD₅₀ of PK-CPT-NP was 120 mg/kg.

2. Each dose for an adult was 50-500 mg. Using 50 kg as the average weight of an adult the dosage of PK-CPT-NP were 1-10 mg/kg, and they are very safe.

3. As to subacute toxicity tests, a dosage corresponding to 50 times the clinical dose of free CPT and PK-CPT-NP was administered continually for two months, and no side effects had been observed. The electrocardiograms and functions of liver and the kidney had not been effect and no injuries whatever had been observed in the tissue slices of the heart, liver, spleen, lungs, kidneys and adrenal.

4. LD₅₀ of PK-CPT-NP is higher than LD₅₀ of CPT. It means that PK-CPT-NP is safer than free CPT.

### EXAMPLE 50

### NP Size

The NP size was measured by light scattering; an average diameter of 80 nm was found. NP size before and after purification is listed as the following table.

TABLE 40

| | |
|---|---|
| PK concentration | Size |

| (% w/v) | Before | After |
|---|---|---|
| 0.125 | 90 ± 10 | 94 ± 11 |
| 0.25 | 80 ± 9 | 83 ± 9 |
| 0.5 | 65 ± 7 | 67 ± 8 |

The data of Table 40 indicated that the purification did not alter NP size. It means that NP is very stable.

### EXAMPLE 51

### Pharmacokinetic Studied of NP

The data of Example 22 indicated that circulation of PK-³H-HHT-NP is longer than ³H-HHT in blood. Present example described Pharmacokinetic studied of NP. Free HHT and PK-HHT-NP were given to mice via the tail at dose of 0.5 mg HHT/kg. The area under the concentration-time curve (AUC) of PK-HHT-NP and free HHT is summarized in the Table 41. Other methods are as same as method of Example 22.

TABLE 41
Tissue of AUC value

| | Tissue AUC (h. μg/g) | | | | | | |
|---|---|---|---|---|---|---|---|
| Formulation | Blood | Liver | Heart | Lung | Spleen | Kidney | Cancer |
| Free HHT | 3.0 | 168.8 | 89.2 | 138.9 | 192.8 | 243.2 | 12.0 |
| PK-HHT-NP | 330.8 | 220.9 | 42.3 | 63.5 | 263.8 | 68.2 | 51.0 |

The data of Table 41 indicated that free HHT was cleared quickly from blood circulation, which is same to the data of Example 22. Leukemia is blood cancer. Therefore, the data of Example 22 and 50 are very important. The AUC of blood for PK-HHT-NP is 100-time higher than free HHT. It means that PK-HHT-NP can make more HHT staying in blood for treatment of leukemia because the blood level of HHT encapsulated in PK-HHT-NP remained high for a long period. In the heart, the HHT concentration was lower compared to free HHT. It is important too, because major side effect of HHT existed in heart.

The data of Table 41 also indicated that PK-HHT-NP could decrease concentration of HHT in heart. It means that HHY-PK-NP could obviously decrease side effect of HHT. The similar results were obtained in lung and kidney. Also, higher levels of PK-HHT-NP in cancer corresponded to the prolonged residence feature of NP. This lung-circulation PK should be useful carriers of chemotherapeutic agents for the treatment of leukemia and solid tumor.

The Pharmacokinetic data of CUR and PK-CUR-NP, ELE and PK-ELE-NP, and CPT and PK-CPT-NP are very similar to the data of HHT and PK-HHT-NP in Example 51.

### EXAMPLE 52

### Preparation of PK-HHT-containing Sterically Stabilized Liposomes (PK-HHT-SSL)

So far, many articles reported drug-containing liposomes. However, general liposomes are not stabilized. Liposomes are stabilized in one month or less. Therefore it is difficult to be used for pharmaceutical industry. In accordance with this invention, PK-HHT-SSL is very stabilized in at least six months. Therefore PK-HHT-SSL can be used in industry. PK-HHT-SSL can enhance cancer targeting and improve anticancer activity of HHT. It is very important that PK improves the characters of liposomes. Also, PK and lipids of soybean are safe for human being. In addition, many articles reported lipids, which used for drug-containing liposomes, are syntheses by organic chemistry. However synthetic lipids have some side effects.

Polysaccharide was extracted from kelp (see Example 2). Hydrogenated phosphatidylcholine (PC), phosphatidylglycerol (PGL), and phosphatidylserine (PS) were extracted from soybean. All above lipids were finally purified on silicic acid columns, shown to be pure by thin-layer chromatography and stored in chloroform in sealed ampoules under nitrogen until use. Phospholipids mixed with cholesterol (CHOL) and long-chain alcohol. The solvent was removed under reduced pressure by a rotory evaporator. The lipids were then purged with nitrogen. Lipids were redissolved in the organic phase and reversed phase will be formed. PK and HHT-containing phosphate-buffered saline (HHT was 3 mM in 0.1 M phosphate-buffered saline) was added at these lipid systems, and resulting two-phase system was sonicated 3 minutes until the mixture homogeneous that did not separate for at least two hours after sonicated. A typical preparation contained 3.3×10⁻³ M of phospholipid and 3.3×10⁻³ M of cholesterol in 1 litre of phosphate-buffered saline, which contained PK, and 3 liters of solvent. PK-HHT-SSL were sealed and sterilized. [³H]-HHT and dialyzed method was used to determine the amount of encapsulated HHT. The size of the vesicles was determined by a dynamic light cattering technique. When PK/PG/PC/CHOL were 3:1:4:5, diameter of liposomes was 20-50 nM ranges. PK-HHT-SSL was very stabilized in at least six months.

### EXAMPLE 53

### Effects of HHT and PK-HHT-SSL on Tumor Cells Proliferation

Materials and Methods

Human tumor cell lines: Hela leukemia HL-60, malignant melanocarcinoma B16, oral epidermoid carcinoma (KB), lung carcinoma (A549), breast carcinoma MCF-7, adenocarcinoma of stomach.

Animal tumor cell lines: Walker carcinoma, LLC-WRC-256, malignant melanoma (RMMI 1846), 3T3, and S-180 sarcoma (CCRF-180). All lines were routinely cultured in the RPMI1640 medium supplemented 20% fetal calf serum. The experiment was carried out in 96 microplate, each well had 5×10⁵ cells and given desired concentration of 1 μg/ml (1×10⁻⁶ g/ml) drug. Then the plate was incubated at 37° C. in an atmosphere of humidified air enriched with 5 percent carbon dioxide for 72 hours.

Inhibition percent rate of tumor cell proliferation was obtained according to the bellow formula.

$$Inhibition percent rate = \frac{Control - Test}{Control} \times 100 \%$$

Results

PK-HHT-SSL and HHT inhibited tumor cells growth significantly.

TABLE 42
Effect of PK-HHT-SSL and HHT on inhibiting
growth cancer cells

Inhibition (%)

| Cell line | HHT | PK-HHT-SSL |
|---|---|---|
| Control | — | — |
| Human cells | | |
| HL-60 | 70.8 ± 9.0 | 86.0 ± 10.8 |
| Hela | 75.6 ± 8.0 | 91.3 ± 10.2 |
| B16 | 73.8 ± 8.1 | 81.6 ± 9.8 |
| KB | 80.0 ± 8.5 | 92.0 ± 11.2 |
| MCF-7 | 78.5 ± 7.9 | 92.8 ± 10.8 |

The data of Table 42 indicated that PK-HHT-SSL and free HHT inhibits growth cancer cells, and the effect of PK-HHT-SSL is better than free HHT.

### EXAMPLE 54

### Anticancer Effect of PK-HHT-SSL

The PK has been demonstrated a strong activity against sarcoma-180 and L-1210, L-615 (dosage is 20 mg/kg/day and inhibitor).

Material and Methods

Animals: Adult DBA/2J male mice, 6 to 8 weeks old. All animals weighed approximately 25 g when used in experiments. Mice were assigned randomly to treatment and control groups. Each member of which received identical dosed (i.p.) of PK-HHT-SSL, HHT or 0.9% NaCl solution for all injections. Volumes were 0.01 ml/g body weight.

Tumor: L1210 or P388 leukemia cells induced in KUM mice by inoculation with L1210 or P388 leukemia cells (1.0×10⁵).

The leukemia cells were passed i.p. weekly. L1210 or P388 cells were cultured at 37° C. in RPMI-1640 medium (GIBCO) without antibiotics and supplemented with 10% fetal calf serum.

After the tumor implantation (1×10⁵ leukemia cells), PK-HHT-SSL was injected intraperitoneally once a day. The treatment was started between the end of 2nd and 6th day after inoculation. Dose of PK-HHT-SSL was 1, 10, 20 mg/kg/day for different animal groups. Control mice were treated with same value of 0-9% NaCl. Anti-leukemia activity was expressed as T/C %. T is median survival days of treated group and C is that of control group. A T/C value was greater that 100% means the treated mice is surviving longer than the control mice. Those rats that survived 60 days after the last day of treatment were considered cured. The statistical significance of data was determined by student's test.

Test data are reported in Table 43-45. PK-HHT-SSL exhibits significant anti-leukemia against the experimental L1210 and P 388 in mice.

TABLE 43
Activity of PK-HHT-SSL against lymphoid leukemia L1210

| Dose of PK (mg/kg/day) | Survival time (days) |
|---|---|
| Untreated | 8.9 ± 0.3 |
| PK-HHT-SSL | 28.9 ± 4.2 |
| HHT | 15.6 ± 2.8 |

TABLE 44
Activity of PK-HHT-SSL against P388 lymphocytic leukemia

| Dose of PK | |
|---|---|
| (mg/kg/day) | Survival time (days) |
| Untreated | 8.9 ± 0.3 |
| PK-HHT-SSL | 34.8 ± 5.9 |
| HHT | 19.8 ± 4.2 |

TABLE 45
Effect of PK-HHT-SSL on sarcoma-180 solid tumors in mice*

| Dose | Number of mice | Mean survival (%)* |
|---|---|---|
| Control | 20 | 5 |
| PK-HHT-SSL | 20 | 72.2 |
| HHT | 20 | 58.6 |

*Percentage survival: 30 days observation after tumor cell inoculation.

The data of Table 43-45 indicated that PK-HHT-SSL and HHT both could obviously inhibit cancer and PK-HHT-SSL is much better and free HHT.

### EXAMPLE 55

### Safety of Composition of PK-HUT-SSL

1. LD₅₀: The LD₅₀ of free HHT in mice (I.P.) was found to be 3.3±0.29 mg/kg.

2. LD₅₀ of PK-HHT-SSL in mice (I.P.) was found to be 6.5±0.68 mg/kg.

3. Each dose for an adult was 50-500 mg. Using 50 kg as the average weight of an adult the dosage of PK-HHT-SSL was 1-10 mg/kg, therefore, it was very safe.

According to above experiments, PK-HHT-SSL increased safety and selectivity to leukemic cells and solid tumors without any loss of HHT efficacy. PK-HHT-SSL reduced HHT toxicity for normal tissues, mainly for the bone marrow and cardiac muscles. PK-HHT-SSL modified the treatment schedules, e.q. and reduced the administered doses. Treating leukemia and tumor activity of PK-HHT-SSL was proved to enhance as compared to the free HHT.

LD₅₀ of PK-HHT-NP is higher than LD₅₀ of free HHT. The above toxicology data mean that PK-HHT-SSL is safer than free HHT.

### EXAMPLE 56

### The Leakage of Liposome

The leakage kinetics of general liposome, which did not have PK, and PK-HHT-SSL were compared. The general liposome meant that it did not contain PK. The results are listed in below table.

TABLE 46

| | Half life of leakage (min) |
|---|---|
| PK-HHT-SSL | 2590.0 ± 322 |
| HHT-SSL | 240.5 ± 40.5 |

The data of Table 46 indicated that half of life of leakage of PK-HHT-SSL is 10-fold more than general liposome. It means that PK can greatly increase stabilization of liposomes.

### EXAMPLE 57

### Effect of PK-HHT-SSL and HHT on Differentiation of Human Leukemic Cells

Methods

Cell Lines. HL-60 cells were established from a patient with acute myeloid leukemia. The cells were cultured in culture flasks with RPMI plus 10% FCS.

Studies of Induction of Differentiation. Differentiation of HL-60 cells was assessed by their abilities to produced superoxide as measured by reduction of NBT, by NSE staining and by morphology as detected on cytospin preparations stained with Diff-Quick stain Set, and by analysis of membrane-bound differentiation markers with two-color immunofluorescence. Briefly, cells were preincubated at 4° C. for 60 min in 10% human AB serum and then with FITC-conjugated mouse IgGI isotype control. Analysis of fluorescence was performed on a flow cytometer.

Cell Cycle Analysis. The cell cycle was analyzed by flow cytometry after 60 h of incubation of HL-60 cells either with or without HHT (10⁻⁸ M) as described. Briefly, the cells were fixed in cold methanol and incubated for 30 min at 4° C. in the dark with a solution of 50 μg/ml propidium iodide, 1 mg/ml Rnase, and 0.1% NP40. Analysis was performed immediately after staining using the CELLFIT program whereby the S-phase was calculated with Rfit model.

Clonogenic Assay in soft Agar. HL-60 cells were culture in a two-layer soft agar system for 10 days without adding any growth factors as described previously, and colonies were counted using an inverted microscope. The analogues were added to the agar upper layer on day 0. For analysis of the reversibility of inhibition of proliferation, the cells were cultured in suspension culture with and without HHT. After 60 h, the culture flasks were gently jarred to loosen adherent cells, the cells were washed twice in cultured medium containing 10% FCS to remove the test drugs, and then the clonogenic assay was performed.

These results were periodically confirmed by fluorescence microscopy and by DNA fragmentation.

TABLE 47
Effect of PK-HHT-SSL and HHT on cellular differentiation of
leukemic cells

| Group | NBT (%) |
|---|---|
| Normal cells | 98 |
| PK-HHT-SSL | 5* |
| HHT | 65* |

*P < 0.001 compared with group of leukemic cells.

The data of Table 47 showed that HHT could significantly induce differentiation of leukemic cells.

### EXAMPLE 58

### Effect of PK-HHT-SSL and HHT on Differentiation of Gastric Cancer Cells

The gastric cancer cells and normal cells were cultured in PRMI 1640 medium supplement with 10% FCS serum. Other method is similar to example 1.

TABLE 48
Effect of HHT on differentiation of gastric cancer cells

| Group | NBT (%) | NSE (%) |
|---|---|---|
| Normal gastric cells | 95 | 92 |
| PK-HHT-SSL | 8 | 5 |
| HHT | 58* | 60* |

*P < 0.001 compared with group of leukemic cells.

The data of Table 48 showed that PK-HHT-SSL and HHT could significantly induce gastric cancer cells. The effect of PK-HHT-SSL is better than free HHT.

### EXAMPLE 59

### Effect of HHT and PK-HHT-SSL on Apoptosis of Cancer Cells

Methods

Human leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml.

Apoptosis Determined by two Methods:

Method (1): Cell pellets containing 5×10⁶ cells were fixed with 2.5% glutaraldehyde in cacodylate buffer (pH 7.4), dehydrated through graded alcohol, and infiltrated with LX-112 epoxy resin. After overnight polymerization at 60° C. 1-μm sections were cut with glass knives using a LKB Nova microtome. The sections were stained with 1% toluidine blue and coverslipped. In addition, experimental examples were stained with May-Grunwald-giemsa stain for the demonstration of apoptosis.

DNA electrophoresis: Untreated and treated HL-60 cells collected by centrifugation, washed in phosphate buffered saline and re-suspended at a concentration of 5×10⁶ cells and 0.1% RNase A. The mixture was incubated at 37° C. for 30 min and then incubated for an additional 30 min at 37° C. with 1 ml protease K. Buffer was added and 25 μl of the tube content transferred to the Horizontal 1.5% agarose gel electrophoresis was performed at 2 V/cm. DNA in gels visualized under UV light after staining with ethidium Bromide (5 μg/ml).

DNA fragmentation assays: DNA cleavage was performed, quantitation of fractional solubilized DNA by diphenylamine assay and the percentage of cells harboring fragmented DNA determined by in labeling techniques. For the diphenylamine assay, 5×10⁶ cells were lysed in 0.5 mL lysis buffer (5 mmol/L Tris-HCl, 20 mmol/L DTA, and 0.5% Triton X-100, pH 8.0) at 4° C. Lysates were centrifuged (15,000 g) for separation of high molecular weight DNA (pellet) and DNA cleavage products (supernatant). DNA was precipitated with 0.5 N perchloric acid and quantitated using diphenylamine reagent. The cell cycle distribution was determined 4 hours after addition of drug and represents mean±SD of 5 independent experiments.

Method (2): Apoptosis of HL-60 cells was assessed by changes in cell morphology and by measurement of DNA nicks using the Apop Tag Kkt (Oncor, Gaithersburg, Md.). Morphologically, HL-60 cells undergoing apoptosis possess many prominent features, such as intensely staining, highly condensed, and/or fragmented nuclear chromatin, a general decrease in overall cell size, and cellular fragmentation into apoptotic bodies. These features make apoptotic cells relatively easy to distinguish from necrotic cells. These changes are detected on cytospin preparations stained with Diff-Quick Stain Set. Apoptotic cells were enumerated in a total of about 300 cells by light microscopy. For evaluation of apoptosis by flow cytometry, cells were fixed and permeabilized in 1% paraformaldehyde and ice-cold 70% ethanol. Digoxigenin-dUTP was incorporated at the 3′OH ends of the fragmented DNA in the presence of terminal deoxynucleotidyltranserase, and the cells were incubated with FITC-labeled anti-digoxigenin-dUTP and with propidium iodide. Green (apoptotic cells) and orange (total DNA) fluorescence were measured with a FACScan flow cytometer and analyzed with LYSIS II and CELLFIT programs.

TABLE 498
Effect of PK-HHT-SSL and HHT on apoptosis of cancer cells

| Drug | Apoptosis (%) |
|---|---|
| Control | 0 |
| HHT | 68.3 ± 7.6 |
| PK-HHT-SSL | 85 ± 9.5* |

*P < 0.001 compared with group of leukemic cells.

The data of Table 49 indicated that HHT-SSL and HHT could significantly induce apoptosis and PK-HHT-SSL is better than HHT.

The preparation of PK-Drug-NP or PK-Drug-SSL, which can be accomplished by the extraction methods set forth above or any conventional methods for extracting the active principles from the plants. The novelty of the present invention resides in the mixture of the active principles in the specified proportions to produce drugs, and in the preparation of dosage units in pharmaceutically acceptable dosage form. The term “pharmaceutically acceptable dosage form” as used hereinabove includes any suitable vehicle for the administration of medications known in the pharmaceutical art, including, by way of example, capsules, tablets, syrups, elixirs, and solutions for parenteral injection with specified ranges of drugs concentration.

In addition, the present invention provides novel methods for treatment of cancer cells with produced safe pharmaceutical agent.

It will thus be shown that there are provided compositions and methods which achieve the various objects of the invention and which are well adapted to meet the conditions of practical use.

As various possible embodiments might be made of the above invention, and as various changes might be made in the embodiments set forth above, it is to be understood that all matters herein described are to be interpreted as illustrative and not in a limiting sense.

What is claimed as new and desired to be protected by Letter Patent is set forth in the appended claims:

1. Polysaccharides of kelp (PK)-anticancer drug-nanoparticles (NP) comprising:
a core formed of PK existing as a solid having function of anticancer and increasing immunity function; and
anticancer drugs, including homoharringtonine (HHT), curcumol (CUR), elemene (ELE) and camptochecin (CPT), surrounding the core, the combination forming a special structure having stronger anticancer effects than free anticancer drug.

2. PK-anticancer drug-NP form of claim 1 wherein said the dosage form includes polysaccharides of kelp (PK) which has anticancer function such as inhibiting leukemia and solid tumor and has increasing immune function of human being and animals such as increasing function of hemopoietic system, increasing blood cells, increasing lymphoblastoid, increasing amount of interleukin, increasing amount of lymphocytes, and increasing CSF and TNF.

3. The natural polysaccharide of claim 1, wherein said producing polysaccharides of kelp (PK) which used for inhibiting cancer and increasing immunity and as polymer for preparation of NP, comprising:
a. finely powdered of kelp was extracted ether and 80% ethanol in order to remove soluble components and residue obtained;
b. the residue was extracted with hot distilled water;
c. water extract was filtered and filtrate saved;
d. ethanol was added to filtrate and precipitate was formed;
e. the precipitates were collected by centrifugation and washed by EtOH and ether, and then dried;
f. the dried powder was frozen overnight and then allowed to thaw at room temperature;
g. the powder was extracted with cold water and removed soluble components;
h. the resulting residue was chromatographed on DEAE-cellulose column and using hot water as elution;
i. the elution was concentrated by evaporation and residue was obtained; and
j. the residue was freeze-dried and final product is polysaccharide is kelp (PK).

4. The anticancer drug of claim 1, wherein said producing homoharringtonine (HHT) which used for treatment of leukemia and solid tumors, comprising:
a. extracting a ground plant selected from the group consisting of Cephalotaxus fortunei Hook, C. sinensis Li, C. hainanensis and C. wilsoniana with 90% ethanol at room temperature for 24 hours;
b. filtering the above mixture and separating a filtrate A from a filter residue;
c. percolating the filter residue with ethanol and collecting a filtrate B;
d. combining filtrates A and B and distilling them under reduced pressure to recover ethanol and an aqueous residue was obtained;
e. acetic acid was added to residue and adjusting the pH of the residue to 2.5;
f. separating solids from the resulting mixture by filtration to yield a filtrate;
g. adjusting the pH of the filtrate of step (f) to 9.5;
h. extracting the alkaline solution of step (g) five times with chloroform, combining all the chloroform extracts and distilling them to recover alkaloids;
i. dissolving the alkaloids in citric acid, and the solution adjusted the pH to 7;
j. the solution of pH 7 was extracted with chloroform;
k. the chloroform was concentrated under reduce pressure and then extracted with buffer of pH 6.7;
l. the chloroform was separated from buffer of pH 6.7 and then extracted with buffer of pH 5;
m. buffer of pH 5 was separated from chloroform;
n. the buffer of pH 5 was adjusted to pH 9 then extracted with chloroform;
o. the chloroform was evaporated under reduced pressure and residue was obtained;
p. the residue was chromatographed on column packed with alumina and using chloroform as elution;
q. the elution (chloroform) was chromatographed on silica gel and using chloroform-buffer of pH 5 as elution;
r. the chloroform-buffer (pH 5) was distilled under reduced pressure and residue obtained;
s. the residue was purified by crystallization in methyl alcohol;
t. crystal was recrystallized in methyl alcohol; and
u. the final product is HHT with 99% purity.

5. The nanoparticles of claim 1, wherein said producing polysaccharide of kelp-homoharringtonine-nanoparticles (PK-HHT-NP) which used for treatment of leukemia and solid tumor, such as lung carcinoma, breast carcinoma, malignant melanocarcinoma, epidermoid and adenocarcinoma of stomach, comprising:
a. PK, HHT and PLA were dissolved in the mixed organic solvent of acetone-dichloromethane;
b. organic solvent was added to poly vinylalcohol (PVA) solution under stirring and emulsion was obtained;
c. emulsion was evaporated under reduced pressure;
d. organic solvent was recovered and NP solidified in aqueous solution;
e. aqueous solution was filtered and filtrate was obtained;
f. filtrate was sedimentated by ultracentrifugation; and
g. sediment was washed twice with ether and dried at room temperature.

6. The nanoparticles of claim 1, wherein said producing homoharringtonine-polysaccharide of kelp-nanoparticles (PK-HHT-NP) which used for treatment of leukemia and solid tumor, such as lung carcinoma, breast carcinoma, malignant melanocarcinoma, epidermoid and adenocarcinoma of stomach, comprising:
a. HHT and PK was added to 1% of dextran solution which containing glucose (pH=3);
b. dextran solution was added corn oil (or cotton seed oil) and poly lysine and then homogenized at 175°-185° C.;
c. the resulting emulsion was then cooled to room temperature and the particles were precipitated by additional ether; and
d. the resulting particles were separated by centrifugation at 10,000 g for 20 minutes and washed twice with additional ether and then dried at room temperature;
e. the final product is PK-HHT-NP.

7. The nanoparticles of claim 1, wherein said producing PK-HHT-NP which used for treatment of leukemia and solid tumor, such as lung carcinoma, breast carcinoma, malignant melanocarcinoma, epidermoid and adenocarcinoma of stomach, comprising:
a. HHT and PK was added to 1% of dextran solution which containing glucose (pH=3);
b. polybutylcyanoacrylate was added to PK-HHT solution with stirring 2 h;
c. the whole dispersed system was filtered and filtrate obtained;
d. filtrate was sedimentated by ultracentrifugation and recovered by removing the water; and
e. the sediment washed twice with ether and then dried at room temperature;
f. the final product is PK-HHT-NP.

8. The nanoparticles of claim 1, wherein said producing PK-HHT-NP which used for treatment of leukemia and solid tumor, such as lung carcinoma, breast carcinoma, malignant melanocarcinoma, epidermoid and adenocarcinoma of stomach, comprising:
a. PK, HHT and gelatin were added to water at 60° C.;
b. oil phase, poly lacticacid and dichloromethane were gradually poured into water phase under vigorous stirring by homogenizer to make an emulsion;
c. the emulsion poured into solution of poly vinylalcohol under stirring;
d. the emulsion was evaporate and dichloromethane was removed;
e. the emulsion was continuously stirred;
f. the NP was collected by filtration;
g. NP washed twice with ether and then dried at room temperature; and
h. the final product is PK-HHT-NP.

9. The anticancer drug of claim 1, wherein said producing curcumol which used for treatment of cancer, comprising:
a. the powder of plants is extracted with water at room temperature;
b. the powder is recovered by filtration;
c. the filtrate is saved and powder of filter residue is extracted with water again;
d. the filtrates are combined and distilled under pressure;
e. the distilled mixture are separated;
f. the oil fraction is saved and kept at 0° C.;
g. the crystals are formed from oil fraction;
h. the crystals are washed with petroleum ether;
i. the needle crystals are obtained after recrystalization from ethanol;
j. the needle crystals are washed with petroleum ether and dried; and
k. the final product is CUR.

10. The anticancer drug of claim 1, wherein said producing elemene which used for treatment and prevention of malignant pleural effusion and cancer and enhancement immune function, comprising:
a. extracting a powder of Drybalanops aromatica Gaerin or Wen E Shu with water;
b. the filtrate was saved and filter residue extracted with water again;
c. the filtrate combined and distilled under pressure;
d. the distilled mixture was separated and oil fraction was kept at 0° C.;
e. the oil distilled under reduced pressure (50°-80° C./40 Pa) and fraction was collected;
f. the fraction was distilled under reduced pressure (76°-78° C./40 Pa) and fraction B was collected;
g. the fraction B was chromatographed on silica gel G and using petroleum ether as developing solvent;
h. the solvent collected and dried; and
i. the final product is ELE.

11. The nanoparticles of claim 1, wherein said producing PK-CUR-NP which used for treatment of cancer, comprising:
a. PK, CUR and poly lactiacid (PLA) dissolved in acetone-dichloromethane mixture;
b. the mixture was poured into aqueous solution of polyvinyl alcohol with stirring using high-speed homogenizer;
c. the whole dispersed system was evaporation in order to remove organic solution;
d. the whole dispersed system was filtered and filtrate was obtained;
e. the filtrate was sedimentated by ultracentrifugation and recovered by removing the water, and
f. the resulting particles washed twice with ether and dried at room temperature.

12. The nanoparticles of claim 1, wherein said producing PK-ELE-NP which used for treatment of cancer, comprising:
a. PK, ELE and poly lactiacid (PLA) dissolved in acetone-dichloromethane mixture;
b. the mixture was poured into aqueous solution of poly vinylalcohol with stirring using high-speed homogenizer;
c. the whole dispersed system was evaporation in order to remove organic solution;
d. the whole dispersed system was filtered and filtrate was obtained;
e. the filtrate was sedimentated by ultracentrifugation and recovered by removing the water, and
f. the resulting particles washed twice with ether and dried at room temperature.

13. The anticancer drug of claim 1, wherein said producing Camptothecine which used for treatment of cancer, comprising:
a. ground Camptotheca acuminata Decne was extracted with ethanol;
b. filtered extracted solution to yield filter residue;
c. filter residue was extracted with CHCl₃;
d. the CHCl₃ solution was filtered and yield filtrate;
e. the filtrate was distilled under reduced pressure to recover CHCl₃ and obtained distilled residue;
f. the distilled residue was extracted with methylic alcohol;
g. the solution of methylic alcohol was distilled under reduced pressure to recover methylic alcohol and distilled residue obtained;
h. the distilled residue was extracted by petroleum ether;
i. filtered the solution to yield filter residue;
j. 10% NaOH was added to filter residue under stirring;
k. solution of NaOH was warmed and filtered at 60° C.;
l. HCl and methylic alcohol was added to solution of NaOH and sediment was obtained;
m. sediment was crystallized with CHCl₃—CH₃OH; and
n. the final product is CPT.

14. the nanoparticles of claim l, wherein said producing PK-CPT-NP which used for treatment of cancer, comprising:
a. PK, CPT and poly lactiacid (PLA) dissolved in acetone-dichloromethane mixture;
b. the mixture was poured into aqueous solution of polyvinyl alcohol with stirring using high-speed homogenizer;
c. the whole dispersed system was evaporation in order to remove organic solution;
d. the whole dispersed system was filtered and filtrate was obtained;
e. the filtrate was sedimentated by ultracentrifugation and recovered by removing the water, and
f. the resulting particles washed twice with ether and dried at room temperature.

15. The natural anticancer drug of claim 1, wherein said the amount sufficient to inhibiting tumor cells proliferation, is about 50-500 mg of PK-HHT-NP.

16. The natural anticancer drug of claim 1, wherein said the amount sufficient to decreasing activity of tyrosine kinase, is about 50-500 mg of PK-HHT-NP.

17. The natural anticancer drug of claim 1, wherein said the amount sufficient to inducing apoptosis, is about 50-500 mg of PK-HHT-NP.

18. The natural anticancer drug of claim 1, wherein said the amount sufficient to inhibiting growth of transplanted tumor, is about 50-500 mg of PK-HHT-NP.

19. The natural anticancer drug of claim 1, wherein said the amount sufficient to increasing anticancer therapeutic index, is about 50-500 mg of PK-CUR-NP.

20. The natural anticancer drug of claim 1, wherein said the amount sufficient to inducing differentiation of cancer cells, is about 50-500 mg of PK-CUR-NP.

21. The natural anticancer drug of claim 1, wherein said the amount sufficient to inhibiting tumor cells proliferation and growth of transplanted tumor, is about 50-500 mg of PK-CUR-NP.

22. The natural anticancer drug of claim 1, wherein said the amount sufficient to inducing apoptosis of cancer cells, is about 50-500 mg of PK-CUR-NP.

23. The natural anticancer drug of claim 1, wherein said the amount sufficient to inhibiting tumor incidence, is about 50-500 mg of PK-CUR-NP.

24. The natural anticancer drug of claim 1 wherein said the amount sufficient to increasing anticancer therapeutic index, is about 50-500 mg of PK-ELE-NP.

25. The natural anticancer drug of claim 1, wherein said the amount sufficient to inducing differentiation of cancer cells, is about 50-500 mg of PK-ELE-NP.

26. The natural anticancer drug of claim 1, wherein said the amount sufficient to inhibiting tumor cells proliferation, is about 50-500 mg of PK-ELE-NP.

27. The natural anticancer drug of claim 1, wherein said the amount sufficient to inducing apoptosis of cancer cells, is about 50-500 mg of PK-ELE-NP.

28. The natural anticancer drug of claim 1, wherein said the amount sufficient to inhibiting growth of transplanted tumor, is about 50-500 mg of PK-ELE-NP.

29. The natural anticancer drug of claim l, wherein said the amount sufficient to inhibiting tumor incidence, is about 50-500 mg of PK-ELE-NP.

30. The natural anticancer drug of claim 1 wherein said the amount sufficient to increasing anticancer therapeutic index, is about 50-500 mg of PK-CPT-NP.

31. The natural anticancer drug of claim 1, wherein said the amount sufficient to inducing differentiation of cancer cells, is about 50-500 mg of PK-CPT-NP.

32. The natural anticancer drug of claim 1, wherein said the amount sufficient to inhibiting tumor cells proliferation, is about 50-500 mg of PK-CPT-NP.

33. The natural anticancer drug of claim 1, wherein said the amount sufficient to inducing apoptosis of cancer cells, is about 50-500 mg of PK-CPT-NP.

34. The natural anticancer drug of claim 1, wherein said the amount sufficient to inhibiting growth of transplanted tumor, is about 50-500 mg of PK-CPT-NP.

35. The natural anticancer drug of claim 1, wherein said the amount sufficient to inhibiting tumor incidence, is about 50-500 mg of PK-CPT-NP.

36. A natural polysaccharide of kelp (PK)-drug-derivates containing sterically stabilized liposomes (PK-drug-SSL) comprising:
a core formed of PK existing as a solid having function of anticancer and increasing immunity function; and
phosphatidylcholine (PC), phosphatidylglycerol (PGL), and phosphatidylserine (PS) combining with core, the combination forming special structure having stronger anticancer effects than free anticancer drug.

37. The anticancer drug of claim 36, wherein said for treating leukemia and solid tumor comprises PK-HHT-SSL.

38. The anticancer drug of claim 36, wherein said Homoharringtonine derivate is extracted from Cephalotaxus sinensis Li or Cephalotaxus hainanensis Li.

39. The anticancer drug of claim 36, wherein said the HHT derivate is Homoharringtonine.

40. The anticancer drug of claim 36, wherein said the HHT derivate is Harringtonine.

41. The anticancer drug of claim 36, wherein said the amount sufficient to induce differentiation of cancer cells to resemble normal cells, is about 50-500 mg of PK-HHT-SSL.

42. The anticancer drug of claim 36, wherein said the amount sufficient to induce apoptosis of cancer cells, is about 50-500 mg of PK-HHT-SSL.

43. The anticancer drug of claim 36, wherein said the amount sufficient to inhibit leukemia cells, is about 25-200 mg of HHT-SSL.

44. The anticancer drug of claim 36, wherein said the amount sufficient to inhibit cancer cells proliferation, is about 50-500 mg of HHT-SSL.

45. The anticancer drug of claim 36, wherein said liposomes contained Hydrogenated phosphatidylcholine (PC), phosphatidylglycerol (PGL), and phosphatidylserine (PS).

46. The PK-Drug-SSL of claim 36, wherein said Hydrogenated phosphatidylcholine (PC), phosphatidylglycerol (PGL), and phosphatidylserine (PS) extracted from soybean.

47. The PK-Drug-SSL of claim 36, wherein said Hydrogenated phosphatidylcholine (PC), phosphatidylglycerol (PGL), and phosphatidylserine (PS) purified on silicic acid columns, shown to be pure by thin-layer chromatography.

48. The PK-Drug-SSL of claim 36, wherein said the amount of encapsulated HHT and HHT-SSL were determined by [³H]-HHT and dialyzed.

49. The PK-Drug-SSL of claim 36, wherein said when PG/PC/CHOL were 1:4:5, diameter of liposomes was about 20-50 nM.

50. The PK-Drug-SSL of claim 36, wherein said the dosage form of PK-HHT-SSL is tablet or capsule form.

51. A dosage unit of claim 36 wherein said dosage form is tablet, including in addition pharmaceutical acceptable binder and excipients.

52. A dosage unit of claim 36 wherein said dosage from is a solution for parenteral injection, which includes in addition a liquid vehicle suitable for parenteral administration.

53. The PK-Drug-SSL of claim 36, wherein said producing PK-HHT-containing sterically stabilized liposomes (PK-HHT-SSL), comprising:
a. PK was extracted from kelp;
b. Phosphatidylcholine (PC), phosphatidylglycerol (PGL), and phosphatidylserine (PS) were purified from soybean;
c. PC, PGL, and PS were purified on silicic acid columns;
d. PC, PGL, and PS mixed with cholesterol (CHOL) and long-chain alcohol;
e. Lipids were dissolved in the organic phase and reversed phase would be formed;
f. PK and HHT solution (HHT 3 mM in 0.1 m phosphate-buffered saline) was added at lipid systems and resulting two-phase system was sonicated; and
g. PK-HHT-SSL was sealed and sterilized.

54. The PK-Drug-NP of claim 36 wherein said liposomes, which contained PK is much stabilized than general liposomes.

55. The PK-Drug-NP of claim 36 wherein said for treatment of cancer comprises PK-CUR-SSL.

56. The PK-Drug-NP of claim 36 wherein said for treatment of cancer comprises PK-ELE-SSL.

57. The PK-Drug-NP of claim 36 wherein said for treatment of cancer comprises PK-CPT-SSL.

58. Polysaccharides of kelp (PK)-active compound-nanoparticles (NP) comprising:
a core formed of PK existing as solid having function of anticancer and increasing immunity; and
the natural polymer, including polysaccharides, serum albumin, gelatin polylysine, poly lacticacid, coating surrounding the core, the combination forming a special structure having more stable particle and stronger effect than free active compound.

59. The PK-active compound-NP of claim 58 has a size of particle is 10-100 nM.

60. The PK-active compound-NP of claim 58 further comprises an active compound within NP.

61. The PK-Drug-NP of claim 60 wherein said the solid core consists the active compound.

62. The PK-active compound-NP of claim 58 wherein said the vehicle is selected from group consisting of natural polymers.

63. The PK-active compound-NP of claim 58 wherein said the active compound is selected from the group consisting of drug, food, additives, pesticides, herbicides, insecticides and pheromones.

64. The PK-HHT-NP of claim 1 and 58 has the following characters: 87.9% of encapsulation and 96.6% of recovery of NP.

65. The PK-active compound-NP of claim 1 and 58, which is suitable for injection into patients.

66. The PK-Drug-NP of claim 1 and 58, which is suitable for capsule or tablet into patients.

67. The PK-HHT-NP of claim 1 and 58, wherein said effect of anticancer chemotherapy of PK-HHT-NP is stronger than free HHT.

68. The PK-CUR-NP of claim 1 and 58, wherein said effect of anticancer chemotherapy of PK-CUR-NP is stronger than free CUR.

69. The PK-ELE-NP of claim 1 and 58, wherein said effect of anticancer chemotherapy of PK-ELE-NP is stronger than free ELE.

70. The PK-CPT-NP of claim 58, wherein said effect of anticancer chemotherapy of PK-CPT-NP is stronger than free CPT.

71. The PK-Drug-NP of claim 1 and 58, wherein said therapeutic effect of PK-Drug-NP is stronger than free drug by PK-Drug-NP delayed clearance anticancer drug from the circulation.

72. The PK-Drug-NP of claim 1 and 58, wherein said therapeutic effect of PK-Drug-NP is stronger than free drug by PK-Drug-NP increased blood circulation time of drug.

73. The PK-Drug-NP of claim 71 and 72, wherein said the drug is HHT.

74. The PK-Drug-NP of claim 71 and 72, wherein said the drug is CUR.

75. The PK-Drug-NP of claim 71 and 72, wherein said the drug is ELE.

76. The PK-Drug-NP of claim 71 and 72, wherein said the drug is CPT.

77. The PK-Drug-NP of claim 58, wherein said PK-Drug-NP is more stable.

78. The PK-Drug-NP of claim 77, wherein said drug is HHT.

79. The PK-Drug-NP of claim 77, wherein said drug is CUR.

80. The PK-Drug-NP of claim 77, wherein said drug is ELE.

81. The PK-Drug-NP of claim 77, wherein said drug is CPT.

82. The PK-HHT-NP of claim 1 and 77, wherein said the amount of encapsulated HHT in PK-HHT-NP could be determined by ³H-HHT and scintillation counter.

83. The PK-CUR-NP of claim 1 and 77, wherein said the amount of encapsulated HHT in PK-CUR-NP could be determined by ³H-CUR and scintillation counter.

84. The PK-ELE-NP of claim 1 and 77, wherein said the amount of encapsulated HHT in PK-ELE-NP could be determined by ³H-ELE and scintillation counter.

85. The PK-CPT-NP of claim 1 and 77, wherein said the amount of encapsulated HHT in PK-CPT-NP could be determined by ³H-CPT and scintillation counter.
